(19) United States
(12) Patent Application Publication
(10) Pub. No.: US 2025/0257018 A1
(43) Pub. Date: Aug. 14, 2025

(54) CO-PRODUCTION OF SUSTAINABLE LOW-CARBON FUELS FROM CO2 AND H2

(71) Applicant: Saudi Arabian Oil Company, Dhahran (SA)

(72) Inventors: Ahmad Khan, Dhahran (SA); Vishvedeep Bhat, Dhahran (SA); Tamour Javed, Dhahran (SA); Md Ashraful Islam, Dhahran (SA); Rashed Mohammad Aleisa, Dhahran (SA)

(73) Assignee: Saudi Arabian Oil Company, Dhahran (SA)

(21) Appl. No.: 18/882,444

(22) Filed: Sep. 11, 2024

Related U.S. Application Data
(60) Provisional application No. 63/551,636, filed on Feb. 9, 2024.

(51) Int. Cl. C07C 1/04; C10G 11/18; C10L 3/10

(52) U.S. Cl. CPC C07C 1/0485; C10G 11/18; C10L 3/103

(57) ABSTRACT

A process for producing low-carbon methanol includes upgrading a natural gas stream in a methanol production unit to produce a methanol reactor effluent and introducing a captured CO₂ feed stream, a low-carbon hydrogen feed stream, or both to a syngas synthesis section or to a syngas stream downstream of the syngas synthesis section and upstream of a methanol reactor of a methanol synthesis section. At least a portion of the methanol reactor effluent includes low-carbon methanol. The low-carbon methanol is a portion of total methanol in the methanol reactor effluent that is attributed to the introducing the captured CO₂ feed stream, the low-carbon hydrogen feed stream, or both to the methanol production unit based on a mass balance certification basis, an energy balance certification basis, or a trace-the-atom certification basis. The low-carbon methanol can be used in an FCC system for producing low-carbon fuels and chemicals.

### CROSS-REFERENCE TO RELATED APPLICATION

This application claims the benefit of priority of U.S. Provisional Patent Application Ser. No. 63/551,636, filed Feb. 9, 2024, which is entitled “CO-PRODUCTION OF SUSTAINABLE LOW-CARBON FUELS FROM CO₂ AND H₂,” the entire contents of which, including the drawings, are hereby incorporated by reference in the present disclosure.

### TECHNICAL FIELD

The present disclosure relates to processes and systems for producing low-carbon fuels and chemicals, and more specifically processes and systems for producing sustainable low-carbon methanol, fuels, and chemicals using co-feeds of low-carbon hydrogen (H₂), captured carbon dioxide (CO₂), or both to existing refinery processes.

### BACKGROUND

As the world ramps up towards more sustainable fuels and chemicals to reach climate neutrality, industry is looking to innovative solutions to produce sustainable products, such as sustainable fuels and chemicals. Currently-available sustainable technologies for producing sustainable fuels and chemicals generally face economic and scalability obstacles, such as significant capital investment and availability of feedstocks, which can impede adoption of these sustainable technologies.

### SUMMARY

A need exists for systems and processes for producing more sustainable fuels and chemicals in a more economically efficient manner. E-fuel refers to a type of synthetic fuel made from renewable energy sources such as but not limited to wind, solar, hydropower, or combinations of these energy sources. E-fuels, synthetic fuels, or both can be produced from carbon provided by carbon dioxide (CO₂) or carbon monoxide (CO) captured from the atmosphere, biogenic, or point sources and hydrogen (H₂) generated from renewable energy or low-carbon sources. These components can be used as feedstocks to produce an array of hydrocarbons through various reactions conducted in various process units. The reactions and process units can be selected to meet certain specifications for fuels and chemicals. For instance, low-carbon gasoline, such as e-gasoline, can be produced using CO₂-to-Methanol and Methanol-to-Gasoline reactions. Another route is CO₂-to-Syngas followed by the Fischer-Tropsch reaction to produce low-carbon middle distillates, such as e-middle distillates. Targeted low-carbon fuels and chemicals production is scalable, but economically challenging, as these processes require significant capital investment and high operational costs due to the handling requirements associated with the unconventional feedstocks.

It is desirable to be able to efficiently generate certifiable low-carbon fuels and chemicals without the expense and investment of a dedicated e-fuel production facility. Accordingly, there is a clear and long-standing need to provide an efficient and economical process for the production of certifiable low-carbon fuels and chemicals (“low-carbon” used to modify fuels and chemicals refers to the carbon footprint of producing the fuels and chemicals and not to the number of carbon atoms in the molecules). The processes and systems of the present disclosure address this clear and long-standing need by utilizing existing assets such as natural gas-based (NG-based) methanol production plants and Fluidized Catalytic Cracking (FCC) units in conventional refineries to minimize capital investment. To generate certifiable low-carbon fuels and chemicals, H₂ produced using low-carbon sources (e.g., sources having low carbon number) or renewable sources, captured CO₂, or both may be introduced to an existing NG-based methanol production unit. Sustainability credit from these alternative feedstocks will propagate further through the sequential products such as low-carbon methanol to low-carbon fuels and chemicals. This novel pathway for the production of sustainable low-carbon fuels requires minimal investment to produce large volumes of products and achieves certifiable low-carbon fuels and chemicals without large capital investment.

According to aspects of the present disclosure, a process for producing low-carbon methanol may comprise upgrading a natural gas stream in a methanol production unit to produce a methanol reactor effluent, wherein the methanol production unit comprises a syngas synthesis section and a methanol synthesis section downstream of the syngas synthesis section; and introducing a captured CO₂ feed stream, a low-carbon hydrogen feed stream, or both to the syngas synthesis section or to a syngas stream downstream of the syngas synthesis section and upstream of a methanol reactor of the methanol synthesis section. At least a portion of the methanol reactor effluent comprises low-carbon methanol. The low-carbon methanol comprises a portion of total methanol in the methanol reactor effluent that is attributed to the introducing the captured CO₂ feed stream, the low-carbon hydrogen feed stream, or both to the methanol production unit based on a mass balance certification basis, an energy balance certification basis, or a trace-the-atom certification basis.

According to another aspect of the present disclosure, a process for producing sustainable fuel and chemical components through fluidized catalytic cracking (FCC) may comprise: catalytically cracking a conventional FCC feed with an FCC catalyst in an FCC reactor to produce an FCC effluent comprising fuel and chemical components; injecting a low-carbon oxygenate stream into the FCC reactor, wherein the low-carbon oxygenate stream comprises oxygenate compounds produced from sustainable sources using captured CO₂, low-carbon hydrogen sources, biologic sources, or combinations thereof; recovering the FCC effluent; and certifying at least a portion of the FCC effluent as low-carbon fuel and chemical components, wherein the low-carbon fuel and chemical components are the fuel and chemical components attributed to injection of the low-carbon oxygenate stream to the FCC reactor based on a mass balance certification basis, an energy balance certification basis, or a trace-the-atom certification basis.

According to still another aspect of the present disclosure, a process for producing sustainable hydrocarbon fuels through fluidized catalytic cracking (FCC) may comprise: producing a low-carbon oxygenate stream comprising low-carbon oxygenate compounds; catalytically cracking a conventional FCC feed stream with a first FCC catalyst in a first FCC reactor to produce a first FCC effluent and used first FCC catalyst; catalytically cracking the low-carbon oxygenate stream with a second FCC catalyst in a second FCC reactor to produce a second FCC effluent and a used second FCC catalyst, wherein the second FCC reactor is parallel to the first FCC reactor; passing the first FCC effluent and the second FCC effluent to an FCC effluent separation system; separating the first FCC effluent and the second FCC effluent in the FCC effluent separation system to produce at least one product stream comprising fuel and chemical components; and certifying at least a portion of the fuel and chemical components as low-carbon fuel and chemical components, wherein the low-carbon fuel and chemical components are the fuel and chemical components attributed to fluidized catalytic cracking of the low-carbon oxygenate stream in the second FCC reactor based on a mass balance certification basis, an energy balance certification basis, or a trace-the-atom certification basis.

Additional features and advantages of the system and methods of the present disclosure will be set forth in the detailed description that follows. The additional features and advantages of the system and methods of the present disclosure will be, in part, readily apparent to those skilled in the art based on the description or recognized by practicing the described embodiments.

### BRIEF DESCRIPTION OF THE DRAWINGS

The following detailed description of specific embodiments of the present disclosure can be best understood when read in conjunction with the following drawings in which:

FIG. 1 schematically depicts a process for producing methanol in natural gas-based methanol production unit, according to embodiments shown and described in the present disclosure;

FIG. 2 schematically depicts a process for generating low-carbon methanol with co-feeding of low-carbon hydrogen and captured CO₂ to a syngas synthesis section of the process of FIG. 1, according to embodiments shown and described in the present disclosure;

FIG. 3 schematically depicts a process for generating low-carbon methanol with introduction of low-carbon hydrogen and captured CO₂ to a syngas at a point upstream of a methanol reactor of the process of FIG. 1, according to embodiments shown and described in the present disclosure;

FIG. 4 schematically depicts a process for generating low-carbon methanol with introduction of low-carbon hydrogen only, without captured CO₂, to various points in the process of FIG. 1, according to embodiments shown and described in the present disclosure;

FIG. 5 schematically depicts a process for generating low-carbon fuels and chemicals through fluidized catalytic cracking with introduction of a low-carbon oxygenate stream as a co-feed, according to embodiments shown and described in the present disclosure; and

FIG. 6 schematically depicts another embodiment of a process for generating low-carbon fuels and chemicals through fluidized catalytic cracking with introduction of a low-carbon oxygenate stream to a dual FCC reactor system, according to embodiments shown and described in the present disclosure.

For the purpose of these simplified schematic illustrations and the present description, the numerous valves, temperature sensors, electronic controllers and the like that are customarily employed and well known to those of ordinary skill in the art of certain refinery operations are not included. Further, accompanying components that are in conventional refinery operations such as, but not limited to, air supplies, nitrogen supplies, catalyst hoppers, flue gas handling, or other pieces of equipment, are not necessarily shown.

It should further be noted that arrows in the drawings refer to process streams. However, the arrows may equivalently refer to transfer lines that may serve to transfer process steams between two or more system components. Additionally, arrows that connect to system components define inlets or outlets in each given system component. The arrow direction corresponds generally with the major direction of movement of the materials of the stream contained within the physical transfer line signified by the arrow. Furthermore, arrows that do not connect two or more system components signify a product stream which exits the depicted system or a system inlet stream which enters the depicted system. Product streams may be further processed in accompanying chemical processing systems or may be commercialized as end products. System inlet streams may be streams transferred from accompanying chemical processing systems or may be non-processed feedstock streams. Some arrows may represent recycle streams, which are effluent streams of system components that are recycled back into the system. However, it should be understood that any represented recycle stream, in some embodiments, may be replaced by a system inlet stream of the same material, and that a portion of a recycle stream may exit the system as a system product.

Additionally, arrows in the drawings may schematically depict process steps of transporting a stream from one system component to another system component. For example, an arrow from one system component pointing to another system component may represent “passing” an effluent from the one system component to another system component, which may include the contents of a process stream “exiting” or being “removed” from one system component and “introducing” the contents of that product stream to another system component.

It should be understood that two or more process streams are “mixed” or “combined” when two or more lines intersect in the schematic flow diagrams. Mixing or combining may also include mixing by directly introducing both streams into a like reactor, separation device, or other system component. For example, it should be understood that when two streams are depicted as being combined directly prior to entering a separator or reactor, in embodiments, the streams could equivalently be introduced into the separator or reactor and be mixed in the reactor. Long dash broken lines are used to identify groupings of components into a system, and short dash broken lines indicate alternative process streams.

Reference will now be made in greater detail to various embodiments, some embodiments of which are illustrated in the accompanying drawings. Whenever possible, the same reference numerals will be used throughout the drawings to refer to the same or similar parts.

### DETAILED DESCRIPTION

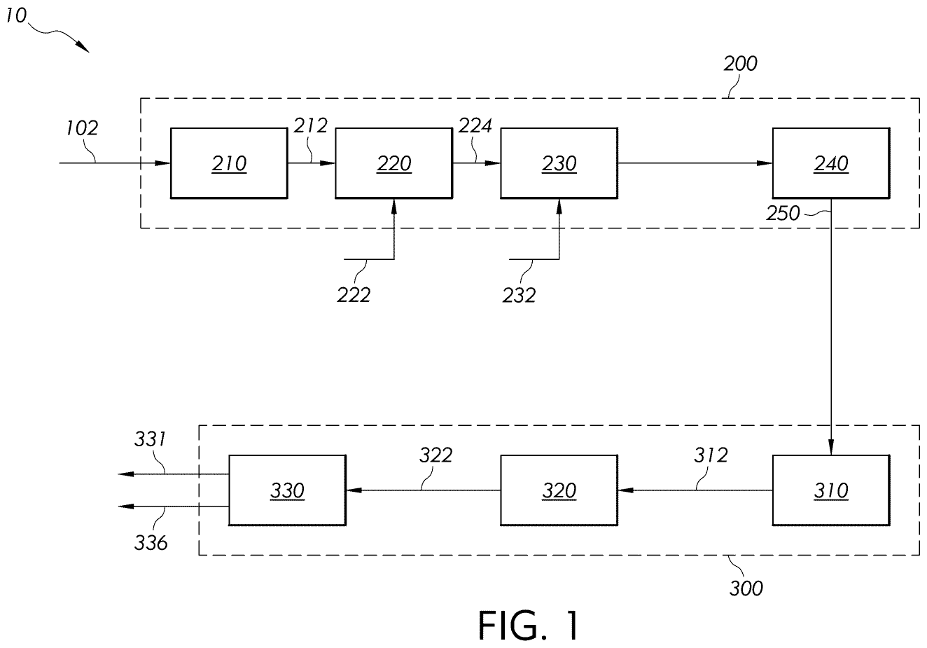

Reference will now be made in detail to embodiments of systems and processes for generating low-carbon fuels and chemicals. While the systems and processes for generating low-carbon fuels and chemicals of FIGS. 1 through 6 are provided as exemplary, it should be understood that the present systems and methods may encompass other configurations. Referring now to FIGS. 1-4, processes of the present disclosure for producing low-carbon methanol may include upgrading a natural gas stream 102 in a methanol production unit 10 to produce a methanol reactor effluent 322, wherein the methanol production unit 10 may comprise a syngas synthesis section 200 and a methanol synthesis section 300 downstream of the syngas synthesis section 200. The processes may further include introducing a low-carbon hydrogen feed stream 110, a captured CO₂ feed stream 120, or both to the syngas synthesis section 200 or to a syngas stream downstream of the syngas synthesis section 200 and upstream of a methanol reactor 320 of the methanol synthesis section 300. At least a portion of the methanol reactor effluent 322 comprises low-carbon methanol 332. The low-carbon methanol 332 is a portion of total methanol in the methanol reactor effluent 322 that is attributed to the introducing of the low-carbon hydrogen feed stream 110, the captured CO₂ feed stream 120, or both to the methanol production unit 10. The low-carbon methanol 332 can be certified as low-carbon methanol on a mass balance certification basis, an energy balance certification basis, or a trace-the-atom certification basis.

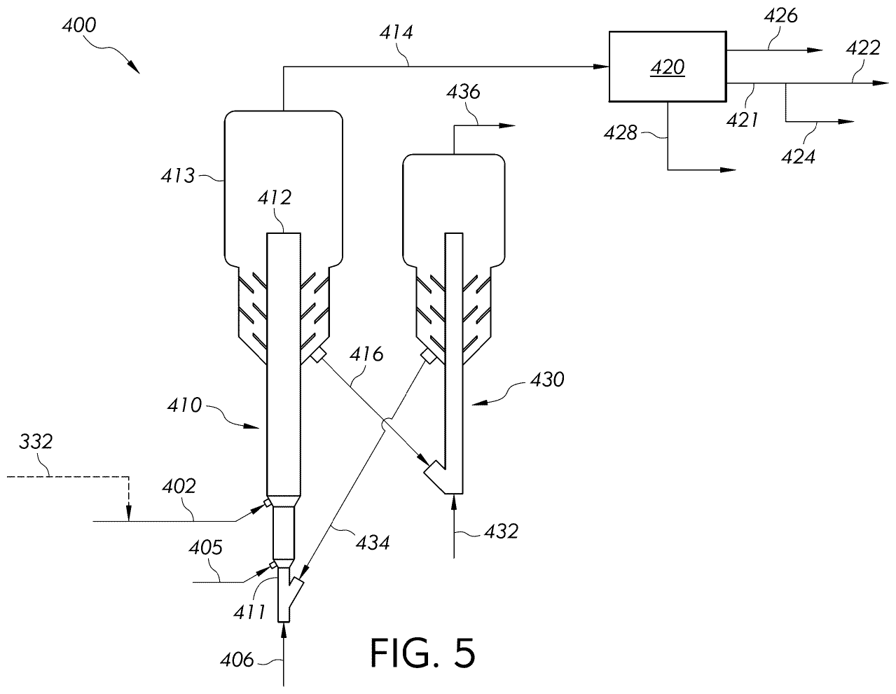

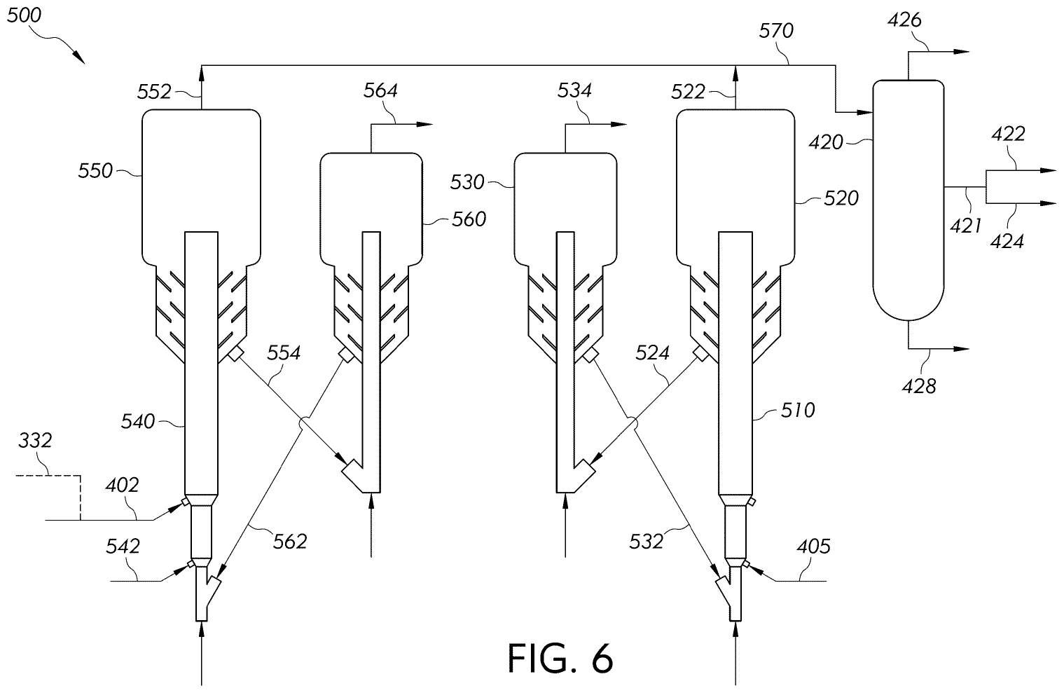

Referring now to FIGS. 5 and 6, methods of the present disclosure for producing sustainable fuel and chemical components through fluidized catalytic cracking (FCC) may include catalytically cracking a conventional FCC feed 405 with an FCC catalyst in an FCC reactor 410 to produce an FCC effluent 414 comprising fuel and chemical components and injecting a low-carbon oxygenate stream 402 into the FCC reactor 410. The low-carbon oxygenate stream 402 may comprise oxygenate compounds produced from sustainable sources using captured CO₂, low-carbon hydrogen sources, biologic sources, or combinations thereof. The methods may further include recovering the FCC effluent 414 and certifying at least a portion of the FCC effluent 414 as low-carbon fuel and chemical components, wherein the low-carbon fuel and chemical components are the fuel and chemical components attributed to injection of the low-carbon oxygenate stream 402 to the FCC reactor 410 based on a mass balance certification basis, an energy balance certification basis, or a trace-the-atom certification basis. In embodiments, the low-carbon oxygenate stream 402 may include the low-carbon methanol stream produced from the methanol production unit 10.

For purposes of this disclosure, it is explicitly noted that indication that one stream or effluent is passed or provided from one unit to another unit includes embodiments where the stream or effluent is passed directly from one unit to another unit as well as embodiments where there is an intervening system or unit which may substantially change the composition of the stream or effluent between the units. As used in the present disclosure, passing a stream or effluent from one unit “directly” to another unit refers to passing the stream or effluent from the first unit to the second unit without passing the stream or effluent through an intervening reaction system or separation system that substantially changes the composition of the stream or effluent. Similarly, indication that two systems are “fluidly connected” indicates that streams may be passed directly between the systems. Heat transfer devices, such as heat exchangers, preheaters, coolers, condensers, or other heat transfer equipment, and pressure devices, such as pumps, pressure regulators, compressors, or other pressure devices, are not considered to be intervening systems that change the composition of a stream or effluent, unless otherwise stated. Combining two streams or effluents together also is not considered to comprise an intervening system that changes the composition of one or both of the streams or effluents being combined. Removing a slip stream from an effluent, where the slip stream has the same composition as the effluent (such as to prevent buildup of contaminants in a process with one or more recycle loops), is also not considered to be an intervening system that changes the composition of the effluent.

It should further be understood that streams may be named for the components of the stream, and the component for which the stream is named may be the major component of the stream (such as a component comprising from 50 weight percent (wt. %), from 70 wt. %, from 90 wt. %, from 95 wt. %, from 99 wt. %, from 99.5 wt. %, or even from 99.9 wt. % of the contents of the stream to 100 wt. % of the contents of the stream, notwithstanding any inert gases, carrier gases, or diluents purposely added to the stream). It should also be understood that components of a stream are disclosed as passing from one system component to another when a stream comprising that component is disclosed as passing from that system component to another. For example, a disclosed “hydrogen stream” passing to a first system component or from a first system component to a second system component should be understood to equivalently disclose “syngas” passing to the first system component or passing from a first system component to a second system component.

The singular forms “a,” “an,” and “the” include plural referents, unless the context clearly dictates otherwise or unless otherwise specifically stated.

Throughout this disclosure ranges are provided. It is envisioned that each discrete value encompassed by the ranges are also included. Additionally, the ranges which may be formed by each discrete value encompassed by the explicitly disclosed ranges are equally envisioned. For brevity, the same is not explicitly indicated subsequent to each disclosed range and the present general indication is provided.

As used in this disclosure and in the appended claims, the words “comprise,” “has,” and “include” and all grammatical variations thereof are each intended to have an open, non-limiting meaning that does not exclude additional elements or steps.

As used throughout the present disclosure, the terms “low-carbon” and “sustainable,” when used in conjunction with a compound or class of compounds, each refer to the compounds or class of compounds being produced by processes or from starting materials having a low-carbon footprint (e.g., low-carbon fuels and chemicals having lower carbon intensity (CI) compared to conventional fuels and chemicals produced from conventional feedstocks (fossil fuels) using conventional non-renewable energy sources (burning of fossil fuels to produce energy)). Unless otherwise stated, the term “low-carbon” does not refer to the number of carbon atoms in a compound. When the number of carbon atoms is material to features herein, the actual number of carbon atoms will be explicitly stated, such as by stating that light olefins refer to olefins having 2-4 carbon atoms.

As used in the present disclosure, the term “e-fuel” refers to fuels or fuel components that are produced from reacting CO₂ and green H₂ produced from water electrolysis powered by renewable energy. Likewise, the term “e-methanol” refers to methanol that is produced from reacting CO₂ and green H₂ produced from water electrolysis powered by renewable energy. It is noted that all e-methanol is low-carbon methanol, but not all low-carbon methanol is e-methanol. The modifier “low-carbon” in front of a species indicates that the species is produced from raw materials or by a process that represents a savings in greenhouse gas emissions over conventional fossil fuel sources and fossil fuel refining methods, whether or not the low-carbon species meets the specifications of certain sustainability standards.

As used in the present disclosure, the term “light olefins” refers to olefins having from 2-4 carbon atoms, such as ethylene, propylene, mixed butenes, and combinations thereof.

As used in the present disclosure, the term “gasoline” refers to hydrocarbons having atmospheric boiling point temperatures of from 25° C. to 221° C.

As used in the present disclosure, the term “middle distillates” refers to hydrocarbons having atmospheric boiling point temperatures of from 221° C. to 343° C.

As previously discussed, the processes and systems of the present disclosure provide a chemical process scheme for generating low-carbon fuels and chemicals. Specifically, the processes and systems of the present disclosure include introducing captured CO₂, low-carbon H₂, or both into existing methanol production units to produce low-carbon methanol, with further processing of this low-carbon methanol or other low-carbon oxygenates to produce more complex low-carbon fuels and chemicals such as gasoline, light olefins, or other compounds.

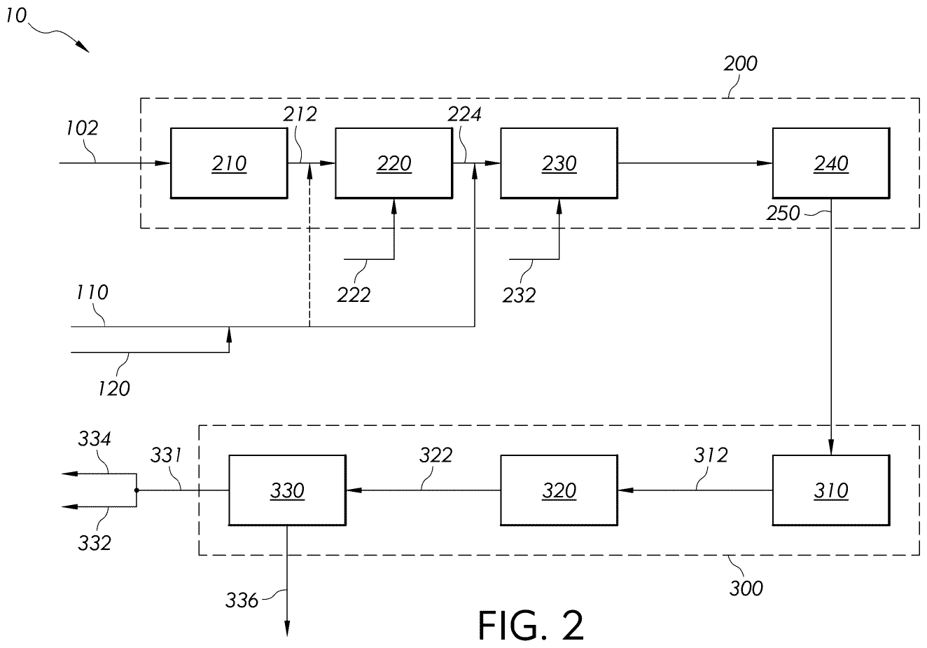

In embodiments, low-carbon methanol is produced in an existing conventional methanol production unit by introducing low-carbon H₂, captured CO₂, or both to the methanol production unit. Referring now to FIG. 1, in a conventional methanol production unit 11, a natural gas (NG) stream 102, which is comprised of mainly methane, is passed as a feedstock to a syngas synthesis section 200 to produce a syngas 250 (e.g., a mixture of CO, CO₂, H₂, H₂O, or combinations thereof) through various processes. The syngas 250 is then passed to a methanol synthesis section 300 to produce conventional methanol 334. Conventional methanol, as used herein, refers to methanol produced from or attributed to the use of conventional fossil-fuel-based raw materials. Referring now to FIGS. 2-4, the systems 100 and processes of the present disclosure include introducing a low carbon H₂ feed stream 110, a captured CO₂ feed stream 120, or both to the methanol production unit 10. In embodiments, the low-carbon H₂ feed stream 110 and the captured CO₂ feed stream 120 may both be introduced to the methanol production unit 10 in a specific molar ratio of the low-carbon hydrogen to the captured CO₂. The low-carbon H₂ feed stream 110, the captured CO₂ feed stream 120, or both may be introduced to and combined with existing process flows upstream of the syngas synthesis section 200, between processing units of the syngas synthesis section 200, or downstream of the syngas synthesis section 200 and upstream of the methanol reactor 320 of the methanol synthesis section 300 to produce low-carbon methanol within the existing methanol production unit 10. Accordingly, the total methanol produced from the methanol production unit 10 would partially comprise low-carbon methanol 332 resulting from the introduction of the low-carbon H₂ 110, the captured CO₂ feed stream 120, or both. As such, a portion of the total methanol produced can be certified as low-carbon methanol, on either a mass balance certification bases, an energy balance certification bases, a trace-the-atom certification basis, or other certification basis, depending on the desired specification requirements. This approach allows for existing methanol production units to be leveraged to generate certifiable low-carbon methanol without significant capital investment to build a stand-alone low-carbon methanol or e-methanol production facility.

In embodiments of the present disclosure, low-carbon fuels and chemicals may also be produced. The system and processes of the present disclosure may include producing low-carbon fuels and chemicals, such as but not limited to olefins, gasoline, or both, by co-cracking the low-carbon methanol or other low-carbon oxygenates in conjunction with conventional or existing feeds in a fluid catalytic cracking (FCC) reactor system. This pathway allows for the transfer of low-carbon credit to the final low-carbon fuels and chemicals produced. Specifically, in a conventional FCC unit, heavy hydrocarbons such as vacuum gas oil, vacuum/atmospheric residue are catalytically cracked to produce lighter hydrocarbons such as but not limited to middle distillates, gasoline, light olefins, light aromatic compounds, or combinations thereof. Referring now to FIGS. 5 and 6, the systems and processes of the present disclosure leverage existing FCC units, such as existing FCC unit 400 in FIG. 5 or existing dual FCC reactor unit 500 in FIG. 6, by co-feeding low-carbon oxygenates 402, such as the low-carbon methanol 332, low-carbon ethanol, or low-carbon dimethyl ether, to the FCC reactors. Referring to FIG. 5, in embodiments, the low-carbon oxygenates 402 may be blended with a conventional FCC feed 405 in the FCC reactor 410 (riser or downer) of the existing FCC unit 400. Referring to FIG. 6, in embodiments, the low-carbon oxygenates 402 may be separately processed in a second FCC reactor 540 (riser or downer) that is separate and distinct from a first FCC reactor 510 that processes the conventional FCC feed 405. Based on the sustainability credits attributed to the low-carbon oxygenates 402 provided to the FCC unit (FCC unit 400 or dual FCC reactor unit 500), a specific amount of the fuels and chemicals produced therefrom may be certified as sustainable low-carbon fuels and chemicals along with the conventional fuels and chemicals. The attribution approach can be either mass-based, energy-based, or trace-the-atom based, as defined under certain recognized certification mechanisms, for example ISCC EU or ISCC Plus, as will be described in further detail in the present disclosure.

### Low-Carbon Hydrogen

Referring to FIGS. 2-4, the low-carbon H₂ feed stream 110 comprises low-carbon hydrogen, which is produced by a process having reduced or no greenhouse gas emissions. The low carbon H₂ in the low-carbon H₂ feed stream 110 may have a smaller carbon footprint compared to hydrogen made from other methods, such as by producing hydrogen from fossil fuels using conventional non-sustainable fuel sources. In embodiments, the low-carbon H₂ in the low-carbon H₂ feed stream 110 can be produced using a variety of methods with little to no greenhouse gas emissions. The low-carbon H₂ source and/or production method for the low-carbon H₂ may be selected to meet specific requirements for the desired specifications for the ultimate product of certified low-carbon fuel or chemical. For instance, if the desired product is e-methanol or e-fuels and chemicals, then “green H₂”, as defined herein, would be required to meet the standards under some standard, such as but not limited to the Renewable Energy Directive (RED II).

In embodiments, water electrolysis powered by renewable electricity may be utilized to generate the low-carbon H₂ in the low-carbon H₂ feed stream 110. Hydrogen generated through water electrolysis powered by renewable energy may be called “green H₂” or “green hydrogen.” It is noted that green hydrogen is considered one of the cleanest and most sustainable hydrogen sources because the green hydrogen is produced entirely by renewable energy, which means production of the hydrogen generates no polluting emissions into the atmosphere. The carbon intensity of green hydrogen depends on the carbon neutrality of the electricity source. The more renewable energy in the electricity mix, the “greener” the hydrogen produced.

In embodiments, hydrogen production from fossil fuels in conjunction with carbon capture and sequestration may be utilized to generate the low-carbon H₂ in the low-carbon H₂ feed stream 110. Hydrogen generated from fossil fuels with carbon capture and sequestration may be called “blue H₂” or “blue hydrogen.” It is noted that blue hydrogen is produced mainly from natural gas, using steam reforming, which brings together natural gas and heated water in the form of steam. Steam reforming of natural gas generates hydrogen, but carbon dioxide is also produced as a by-product which may be captured and sequestered. The carbon capture and sequestration in the production of the blue hydrogen reduces the carbon footprint compared to producing hydrogen from steam reforming of natural gas without carbon capture and sequestration.

In embodiments, water electrolysis powered by nuclear energy may be utilized to generate the low-carbon H₂ in the low-carbon H₂ feed stream 110. Hydrogen generated with nuclear energy-based water electrolysis may be called “pink H₂” or “pink hydrogen” as well as “purple H₂” or “red H₂.” Pink hydrogen is considered a low-carbon H₂ because the process of making pink hydrogen does not produce carbon dioxide emissions during production. The pink hydrogen has a low-carbon footprint due to the zero carbon emissions of the nuclear energy source.

In embodiments, hydrogen production from methane pyrolysis may be utilized to generate the low-carbon H₂ in the low-carbon H₂ feed stream 110. Hydrogen generated from methane pyrolysis may be called “turquoise H₂” or “turquoise hydrogen.” It is noted that turquoise hydrogen is produced by splitting methane in a reactor or blast furnace into hydrogen and solid carbon. It is noted that the low-carbon nature of turquoise hydrogen is dependent on powering the pyrolysis with clean energy and storing the physical carbon generated. In various further embodiments, the low-carbon H₂ in the low-carbon H₂ feed stream 110 may be generated from flue gases, waste gases, or both with low-carbon emissions, such as by recovering hydrogen from the waste gas, flue gases, or both.

In embodiments, hydrogen production from H₂S splitting may be utilized to generate the low-carbon H₂ in the low-carbon H₂ feed stream 110. It is noted that H₂S is generally a waste stream produced from different processes including oil and gas treatment. H₂S can be valorized through the splitting of H₂S to produce H₂ and sulfur solid. The low-carbon nature of this hydrogen derived from splitting H₂S is dependent on powering the H₂S splitting process with clean energy and storing the physical sulfur generated.

In embodiments, the low-carbon H₂ feed stream 110 may include one or more of the following: green hydrogen produced from water electrolysis; blue hydrogen produced from fossil fuels with carbon capture and sequestration; pink hydrogen produced from water electrolysis powered by nuclear energy; turquoise hydrogen produced from recovery of hydrogen from methane pyrolysis; hydrogen produced from H₂S splitting; hydrogen produced from flue gas, waste gas, or both with low-carbon emissions; or combinations of these sources of low-carbon H₂. In embodiments, the low-carbon H₂ feed stream 110 may consist of or consist essentially of green hydrogen produced from water electrolysis. When green hydrogen is used as the low-carbon H₂ feed stream 110 the certifiable portions of the products produced may be certified as e-products, such as e-methanol, e-fuels, e-chemicals, and the like.

In embodiments, the methods and processes disclosed herein may include producing the low-carbon H₂ feed stream 110. Producing the low-carbon H₂ feed stream 110 may include one or more of the following: subjecting water to electrolysis using energy derived from renewable energy sources (wind, solar, etc.) or nuclear power to produce green hydrogen or pink hydrogen; converting fossil fuels to hydrogen and carbon compounds with carbon capture and sequestration to produce blue hydrogen; recovering hydrogen from a methane pyrolysis process to produce turquoise hydrogen; recovering hydrogen from flue gas or waste gas with low-carbon emissions; producing hydrogen from H₂S splitting; or any combinations of these processes for produce low-carbon H₂.

### Captured Carbon Dioxide

Referring again to FIGS. 2-4, the captured CO₂ feed stream 120 comprises carbon dioxide which has been captured to reduce overall CO₂ release into the environment. In embodiments, the carbon dioxide in the captured CO₂ feed stream 120 may be obtained from direct capture from the atmosphere (direct air capture), from biogenic sources, from industrial point sources, or combinations of these sources. In embodiments, carbon dioxide may be directly captured from the air or atmosphere to provide the carbon dioxide in the captured CO₂ feed stream 120. Capturing existing carbon dioxide from the air or atmosphere does not contribute to additional release or generation of carbon dioxide.

In embodiments, carbon dioxide may be captured from biogenic sources to provide the carbon dioxide in the captured CO₂ feed stream 120. Biogenic CO₂ is defined as CO₂ generated as part of the natural carbon cycle, as well as those resulting from the combustion, harvest, digestion, fermentation, decomposition, or other processing of biologically based materials. Biogenic CO₂ is part of the natural carbon cycle where plants absorb CO₂ through photosynthesis and, when they decompose or are otherwise broken down, they release the CO₂ back into the atmosphere. Capturing the generated CO₂ from plant decomposition, combustion, or other processing beneficially utilizes the CO₂ which was released as part of the natural carbon cycle.

In embodiments, carbon dioxide may be captured from industrial point sources to provide the carbon dioxide in the captured CO₂ feed stream 120. Industrial point sources refer to single, identifiable sources of pollution that emit a significant amount of pollutants, such as CO₂. Point sources can include large facilities that emit pollutants, such as CO₂, during manufacturing, power generation, heating, incineration, or other activities. Capturing CO₂ generated from an industrial point source and utilizing it in accordance with the present disclosure for generating low-carbon fuels and chemicals may result in net zero additional release of CO₂. Specifically, the industrial point source, as a result of normal operation, releases the CO₂ which may be captured and utilized to produce the low-carbon fuels and chemicals by the processes of the present disclosure. In certain use cases of the final low-carbon product, using CO₂ from industrial point sources may result in reusing the emitted CO₂ from point source to valuable fuels before it is released into the air again after combustion. The nature of the CO₂ emission over the entire life cycle of the low-carbon produce will depend on the final utilization of the product.

### Low-Carbon Methanol Production

Production of low-carbon methanol in accordance with the various systems and processes of the present disclosure may allow for utilization of existing methanol production units, which can be based on a variety of different processes. Specifically, conventional methanol production may be achieved in processing plants which utilize different reaction routes. In a typical methanol production plant, natural gas is processed to generate syngas which then may be synthesized into methanol in a methanol synthesis reactor, which is typically a fixed catalyst bed type of reactor. As previously discussed, the systems and process of the present disclosure include introducing the low-carbon H₂ in the low-carbon H₂ feed stream 110, the captured CO₂ in the captured CO₂ feed stream 120, or both as co-feeds to an existing feedstock, such as a natural gas stream, at various locations of an existing conventional methanol production unit. Specifically, for the production of low-carbon methanol from a sustainable feedstock, different locations to co-feed the low-carbon H₂ and captured CO₂ are envisioned within the scope of the present disclosure and the various embodiments. The choice of the feeding location into the conventional methanol production process may depend on the original configuration of the plant, existing feedstock composition, the chosen operating conditions of the process units, or any combination of these considerations.

Any existing methanol production unit within a hydrocarbon production facility may be leveraged within the various embodiments of the processes and systems of the present disclosure for generating low-carbon methanol. For purposes of clarity and conciseness, the systems and processes of the present disclosure will be described in the context of the existing methanol production unit 10 illustrated in FIG. 1. The methanol production unit illustrated in FIG. 1 serves as the base methanol production unit which is leveraged and built upon in FIGS. 2, 3, and 4, but it will be appreciated that other types of methanol production units within a hydrocarbon production facility may be leveraged within the various embodiments by providing co-feeds in a substantially similar matter as disclosed in FIGS. 2, 3, and 4.

Referring again to FIG. 1, an example of a methanol production unit 10 within a hydrocarbon production facility may include a syngas synthesis section 200 and a methanol synthesis section 300 downstream of the syngas synthesis section 200. In embodiments, the methanol production unit 10 may be an existing methanol production unit in an existing hydrocarbon production facility. The syngas synthesis section 200 may be configured to convert constituents of a natural gas stream 10 into syngas for subsequent synthesis into methanol in the methanol synthesis section 300. The syngas synthesis section 200 may include a hydrodesulfurization unit 210, a primary reformer 220, a secondary reformer 230, and a heat recovery unit 240. The effluent of the syngas synthesis section 200 is a syngas stream 250, which may be utilized for subsequent processing into methanol in the methanol synthesis section 300. The methanol synthesis section 300 may include a syngas compression unit 310, a methanol reactor 320 to generate methanol, and a methanol purification unit 330 configured to separate methanol from other reaction products and unreacted syngas.

The natural gas stream 102 may generally comprise methane gas (CH₄) as well as other light hydrocarbon and non-hydrocarbon gases, depending on the source of the natural gas. In embodiments, the natural gas stream 102 may include greater than or equal to 70 wt. %, greater than or equal to 80 wt. %, greater than or equal to 90 wt. %, or even greater than or equal to 95 wt. % methane based on the total mass flow rate of the natural gas stream 102. The natural gas stream 102 may also include small amounts of light hydrocarbon gases (ethane, propane, butane and other light hydrocarbon gases), light oxygenates such as methanol, carbon dioxide, nitrogen, hydrogen sulfide, helium, hydrogen, or other light gases that are naturally-occurring in subterranean natural gas sources.

The natural gas stream 102 is introduced to the syngas synthesis section 200 of the methanol production unit 10 to produce the syngas stream 250. As previously discussed, the syngas synthesis section 200 may comprise the hydrodesulfurization unit 210, the primary reformer 220 downstream of the hydrodesulfurization unit 210, the secondary reformer 230 downstream of the primary reformer 220, and the heat recovery unit 240 downstream of the secondary reformer 230. In embodiments, the natural gas stream 102 may be fed into the hydrodesulfurization unit 210 comprising a hydrodesulfurization catalyst. In embodiments, a hydrogen stream (not shown) may also be introduced to the hydrodesulfurization unit 210. In embodiments, the hydrogen stream to the hydrodesulfurization unit 210, when present, may comprise at least a portion of the low-carbon H₂ feed stream 110. The hydrodesulfurization unit 210 may utilize a catalytic chemical process to remove sulfur from the natural gas stream 102. The hydrodesulfurization unit 210 may be operable to contact the natural gas stream 102 with the hydrodesulfurization catalyst, with or without hydrogen, to produce a desulfurized natural gas stream 212, which may have a reduced concentration of sulfur and sulfur compounds compared to the natural gas stream 102. The hydrodesulfurization unit 210 may be configured to remove greater than or equal to 50%, greater than or equal to about 70%, greater than or equal to 80%, or greater than or equal to 90% of the sulfur compounds from the natural gas stream 102 on a molar basis.

The desulfurized natural gas stream 212 may then be reformed to produce the syngas stream 250. Reforming of natural gas to produce syngas can be achieved using a variety of processes including steam methane reforming (SMR), autothermal reforming (ATR), partial oxidation (POX), water gas shift reaction (WGS), or various combinations of these processes. The types of processes for reforming natural gas and the arrangement of the different processes in the syngas synthesis section 200 may be selected to produce a specific syngas composition. In embodiments, the syngas synthesis section 200 may include the primary reformer 220 downstream of the hydrodesulfurization unit 210, and the secondary reformer 230 downstream of the primary reformer 220. The primary reformer 220, the secondary reformer 230, or both may include one or more of a steam methane reforming unit, an autothermal reforming unit, a partial oxidation unit, a water gas shift reaction unit, of any combinations thereof.

Referring again to FIG. 1, the desulfurized natural gas stream 212 may be passed from the hydrodesulfurization unit 210 to the primary reformer 220. In embodiments, the primary reformer 220 may operate on the principals of steam methane reforming. In other words, the primary reformer 220 may include a steam methane reforming unit. In embodiments, the desulfurized natural gas stream 212 and steam 222 may be passed to the primary reformer 220 and contacted in the primary reformer 220 under reforming reaction conditions to produce a primary reformer outlet stream 224.

The primary reformer outlet stream 224 may then be passed to the secondary reformer 230, which is disposed downstream of the primary reformer 220. In embodiments, the secondary reformer 230 may operate on the principals of autothermal reforming. As such, in embodiments, an oxygen stream 232 may also be passed to the secondary reformer 230 or combined with the primary reformer outlet stream 224 upstream of the secondary reformer 230. The secondary reformer 230 may be configured to contact the primary reformer outlet stream 224 with the oxygen of the oxygen stream 232 at autothermal reforming reaction conditions to conduct autothermal reforming of constituents of the primary reformer outlet stream 224 to produce the syngas stream 250 comprising the syngas.

Referring again to FIG. 1, the syngas stream 250 may then be passed from the secondary reformer 230 to the heat recovery unit 240 disposed downstream of the secondary reformer 230. The heat recovery unit 240 may include one or more heat transfer devices configured to capture thermal energy from the syngas stream 250 exiting the secondary reformer 230 for beneficial energy reutilization. The heat recovery unit 240 may recover heat from the syngas stream 250 before passing the syngas stream 250 downstream to the methanol synthesis section 300.

Each of the hydrodesulfurization unit 210, the primary reformer 220, the secondary reformer 230, and the heat recovery unit 240 are not discussed in significant detail with respect to reaction conditions, catalysts, reactor configurations, etc., as these types of processes are processes with which one skilled in the art has extensive familiarity.

The syngas stream 250 may include a syngas comprising CO₂, carbon monoxide (CO), and hydrogen. The syngas stream 250 may further include unreacted constituents, such as unreacted or excess steam (H₂O) and oxygen introduced to the primary reformer 220 or secondary reformer 230, respectively; unreacted methane from the natural gas stream 102; other constituents from the natural gas stream 102; or other compounds produced in the primary reformer 220, secondary reformer 230, or both. The syngas stream 250 may be characterized by a syngas molar ratio, which is defined as [(moles H₂)−(moles CO₂)]/[(moles CO)+(moles CO₂)], based on Reactions 1-3. In embodiments, the syngas stream 250 may have concentrations of H₂, CO, and CO₂ resulting in a syngas molar ratio of from 1.8 to 2.4, from 1.8 to 2.2, from 1.9 to 2.4, from 1.9 to 2.2, from 2.0 to 2.4, or from 2.0 to 2.2.

With continued reference to FIG. 1, the methanol synthesis section 300 may receive the syngas stream 250 from the syngas synthesis section 200 and may be configured to convert at least a portion of the syngas stream 250 into methanol. The methanol synthesis section 300 may include a syngas compression unit 310, a methanol reactor 320 to generate methanol from the syngas, and a methanol purification unit 330 to separate methanol from other products. In embodiments, the syngas stream 250 may be passed to the syngas compression unit 310, which pressurizes and compresses the syngas stream 250 to produce a pressurized syngas stream 312. In embodiments, all of the syngas stream 250 is passed to the methanol synthesis section 300, such as to the syngas compression unit 310, so that none of the syngas stream 250 is passed outside of the process limits of the methanol production unit 10. The syngas compression unit 310 may be disposed downstream of the secondary reformer 230 and upstream of the methanol reactor 320, such as downstream of the heat recovery unit 240 and upstream of the methanol reactor 320. Processes disclosed herein may include compressing the syngas stream 250 in the syngas compression unit 310 to produce the pressurized syngas stream 312, which may then be passed to the methanol reactor 320. In embodiments, all of the syngas stream 250 is passed to the syngas compression unit 310 and compressed to form the pressurized syngas stream 312.

Referring still to FIG. 1, the pressurized syngas stream 312 may then be passed to the methanol reactor 320 to produce methanol. In embodiments, all of the pressurized syngas stream 310 is passed to the methanol reactor 320 so that none of the pressurized syngas stream 310 is passed outside of the methanol production unit 10. In the methanol reactor 320, the syngas of the pressurized syngas stream 312 may be converted to methanol (CH₃OH) according to Reaction 1.

CO+2H₂↔CH₃OH Reaction 1

The reaction of CO and H₂ to produce methanol according to Reaction 1 is an exothermic reaction that occurs at high temperatures and pressures. The methanol reactor 320 may include one or more catalysts to further the reaction to generate methanol. CO₂ may also react with H₂ to produce methanol and water according to Reaction 2.

CO₂+3H₂↔CH₃OH+H₂O Reaction 2

Additionally, CO may react with water (H₂O) in the methanol reactor 320 to produce CO₂ and H₂ according to Reaction 3.

CO+H₂O ↔CO₂+H₂ Reaction 3

The methanol reactor 320 may produce a methanol reactor effluent 322 comprising the methanol produced in the methanol reactor 320 along with any of the unreacted CO, CO₂, and H₂; water produced in the methanol reactor 320; and any constituents from the pressurized syngas stream 312 passing through the methanol reactor 320.

Referring again to FIG. 1, the methanol reactor effluent 322 may be passed from the methanol reactor 320 to the methanol purification unit 330 disposed downstream from the methanol reactor 320. The methanol purification unit 330 may be configured to separate methanol from other products, unreacted species, or combinations thereof to produce a methanol product stream 331 and other constituents 336. The other constituents 336 may be further separated into different streams, which may be either recycled back to the syngas synthesis section 200, recycled back to the methanol synthesis section 300, or otherwise processed outside of the methanol production unit 10. Each of the syngas compression unit 310, the methanol synthesis unit 320, and the methanol purification unit 330 are not discussed in significant detail as they represent units which one skilled in the art has extensive familiarity. Specifically, the generation of methanol from a feed of syngas may be in accordance with any processing scheme known to one skilled in the art and provided as existing units within a hydrocarbon processing facility.

The methanol production unit 10 of FIG. 1 may be used in a process to upgrade the natural gas stream 102 to methanol. The process may include hydrodesulfurizing the natural gas stream 102 in the hydrodesulfurization unit 210 to produce the desulfurized natural gas stream 212; reforming the desulfurized natural gas stream 212 in the presence of steam 222 in the primary reformer 220 downstream of the hydrodesulfurization unit 210 to produce the primary reformer outlet stream 224; reforming the primary reformer outlet stream 224 in the presence of oxygen in the secondary reformer 230 downstream of the primary reformer 220 to produce the syngas stream 250 comprising at least CO₂, CO, and H₂; and converting the syngas stream 250 in the methanol reactor 320 disposed downstream of the secondary reformer 230 to produce the methanol reactor effluent 322. The processes may further include compressing the syngas stream 250 in the syngas compression unit 310 upstream of the methanol reactor 320 to produce the pressurized syngas stream 312 and passing the pressurized syngas stream 312 to the methanol reactor 320.

Referring now to FIGS. 2-4, according to embodiments of the present disclosure, low-carbon methanol 332 can be produced in the existing methanol production unit 10 by introducing the low-carbon H₂ feed stream 110, the captured CO₂ feed stream 120, or both to the existing methanol production unit 10 upstream of the methanol reactor 320. The processes disclosed herein may include introducing the low-carbon H₂ feed stream 110, the captured CO₂ feed stream 120, or both to the syngas synthesis section 200 or to the syngas stream 250 downstream of the syngas synthesis section 200 and upstream of the methanol reactor 320 of the methanol synthesis section 300. A portion of total methanol in the methanol reactor effluent 322 can be certified as low-carbon methanol 332. In embodiments, the processes disclosed herein may include introducing the low-carbon H₂ feed stream 110 and the captured CO₂ feed stream 120 to the syngas synthesis section 200 or to the syngas stream 250 downstream of the syngas synthesis section 200 and upstream of the methanol reactor 320. The reactions to produce methanol from the low-carbon H₂ in the low-carbon H₂ feed stream 110 and the captured CO₂ in the captured CO₂ feed stream 120 are provided in Reactions 1-3.

The processes of the present disclosure for producing the low-carbon methanol in the methanol production unit 10 may be a net consumer of carbon dioxide. Although CO₂ may be recovered from the methanol reactor effluent 322 and recycled back to the syngas synthesis section 200 or to the methanol synthesis section 300, the processes disclosed herein may not include or require any downstream capture or sequestration of carbon dioxide.

Referring now to FIG. 2, in embodiments, the low-carbon H₂ in the low-carbon H₂ feed stream 110 and the carbon dioxide in the captured CO₂ feed stream 120 may be fed into a reformer in the syngas synthesis section 200. In embodiments, the low-carbon H₂ in the low-carbon H₂ feed stream 110 and the carbon dioxide in the captured CO₂ feed stream 120 may be combined with the primary reformer outlet stream 224 of the primary reformer 220 and fed into the secondary reformer 230. In embodiments, the low-carbon H₂ feed stream 110 and the captured CO₂ feed stream 120 may be passed separately and independently to the secondary reformer 230. Feeding the low-carbon H₂ and the captured CO₂ into the secondary reformer 230 may generate CO and H₂O through reaction of the H₂ from the low-carbon H₂ feed stream 110 and CO₂ from the captured CO₂ feed stream 120. Accordingly, the composition of the syngas stream 250 may be controlled by controlling the flow rates of the low-carbon H₂ feed stream 110, the captured CO₂ feed stream 120, or both. The syngas stream 250 may then be processed in the methanol synthesis section 300 to produce methanol. Since the low-carbon H₂ in the low-carbon H₂ feed stream 110 and the CO₂ in the captured CO₂ feed stream 120 represent sustainable feeds, which were utilized to generate a portion of the syngas stream 250 fed to the methanol synthesis section 300, the methanol stream 331 produced from the methanol synthesis section 300 includes both conventional methanol 334 and low-carbon methanol 332. In embodiments, the low-carbon H₂ feed stream 110 may be green hydrogen, and the low-carbon methanol 332 may be certified as e-methanol.

In embodiments, the low-carbon H₂ in the low-carbon H₂ feed stream 110 and the carbon dioxide in the captured CO₂ feed stream 120 may be combined with the desulfurized natural gas stream 212 downstream of the hydrodesulfurization unit 210 and upstream of the primary reformer 220 and then fed into the primary reformer 220. In embodiments, the low-carbon H₂ feed stream 110 and the captured CO₂ feed stream 120 may be passed separately and independently to the primary reformer 220. This arrangement is indicated by the small dash broken line in FIG. 2. Feeding the low-carbon H₂ feed stream 110 and the captured CO₂ feed stream 120 into the primary reformer 220 processes the feeds in conjunction with the desulfurized natural gas stream 212 and generates CO and H₂O through reaction of the H₂ from the low-carbon H₂ feed stream 110 and CO₂ from the captured CO₂ feed stream 120. Accordingly, the composition of the syngas stream 250 may be controlled by controlling the flow rates of the low-carbon H₂ feed stream 110, the captured CO₂ feed stream 120, or both to the primary reformer 220. The syngas stream 250 may then be processed in the methanol synthesis section 300 to produce methanol. Since the low-carbon H₂ in the low-carbon H₂ feed stream 110 and the CO₂ in the captured CO₂ feed stream 120 represent sustainable feeds, which were utilized to generate a portion of the syngas stream 250 fed to the methanol synthesis section 300, the methanol stream 331 produced from the methanol synthesis section 300 includes both conventional methanol 334 and low-carbon methanol 332. In embodiments, the low-carbon H₂ feed stream 110 may be green hydrogen, and the low-carbon methanol 332 may be certified as e-methanol.

The processes of the present disclosure may include introducing the low-carbon H₂ feed stream 110, the captured CO₂ feed stream 120, or both to the syngas synthesis section 200 of the methanol production unit 10. In embodiments, the syngas synthesis section 200 may comprise the primary reformer 220 and the secondary reformer 230 downstream of the primary reformer 220, and the processes may comprise introducing the low-carbon H₂ feed stream, the captured CO₂ feed stream, or both to the syngas synthesis section 200 upstream of the primary reformer 220 or downstream of the primary reformer 220 and upstream of the secondary reformer 230. In embodiments, the processes may include introducing the low-carbon H₂ feed stream 110, the captured CO₂ feed stream 120, or both directly to the primary reformer 220, directly to the secondary reformer 230, or directly to the primary reformer 220 and the secondary reformer 230. In embodiments, the processes may include combining the low-carbon H₂ feed stream 110, the captured CO₂ feed stream 120, or both with the desulfurized natural gas stream 212 upstream of the primary reformer 220, with the primary reformer outlet stream 224 upstream of the secondary reformer 230, or with both the desulfurized natural gas stream 212 and the primary reformer outlet stream 224.

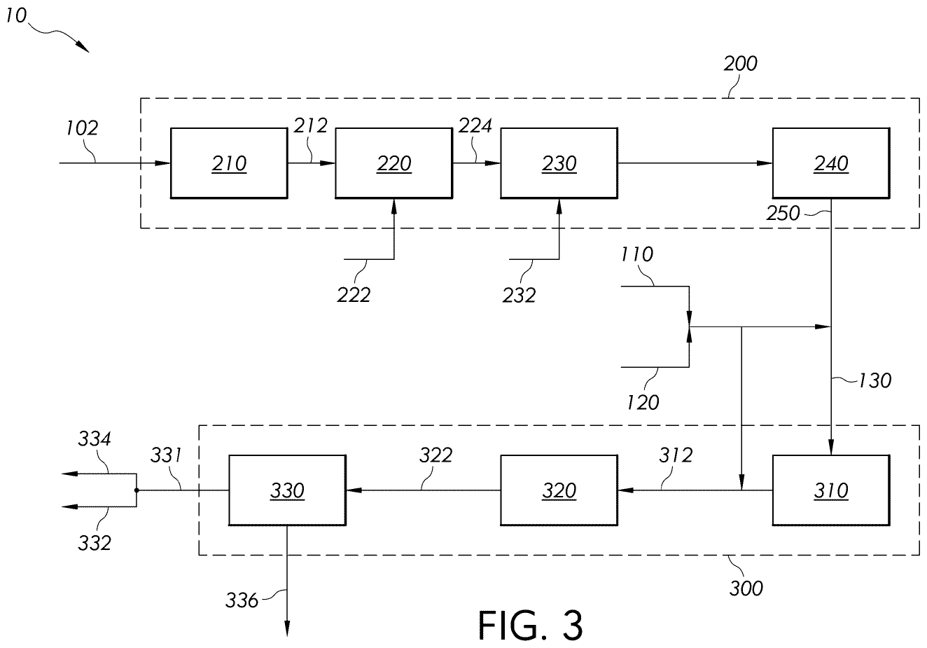

Referring now to FIG. 3, in embodiments, the low-carbon H₂ in the low-carbon H₂ feed stream 110 and the CO₂ in the captured CO₂ feed stream 120 may be combined with the syngas stream 250 produced according to any scheme within the existing methanol production plant. The low-carbon H₂ feed stream 110 and the captured CO₂ feed stream 120 may be introduced to the methanol production unit 10 downstream of the secondary reformer 230 and upstream of the methanol reactor 320. In embodiments, the low-carbon H₂ feed stream 110 and the captured CO₂ feed stream 120 may be combined with the syngas stream 250 downstream of the heat recovery unit 240 to produce a combined syngas stream 130. The combined syngas stream 130 formed from the syngas stream 250, the low-carbon H₂ feed stream 110, and the captured CO₂ feed stream 120 may then be fed to the methanol synthesis section 300 of the existing methanol production unit 10. In embodiments, combined stream 130 may be fed into the syngas compression unit 310 to be pressurized before transfer to the methanol reactor 320 to produce methanol. In embodiments, the low-carbon H₂ feed stream 110 and the captured CO₂ feed stream 120 may be introduced to the methanol synthesis section 300 downstream of the syngas compression unit 310 and upstream of the methanol reactor 320. In embodiments, the low-carbon H₂ feed stream 110 and the captured CO₂ feed stream 120 may be combined with the pressurized syngas stream 312 downstream of the syngas compression unit 310 and upstream of the methanol reactor 320.

The methanol reactor effluent 322 from the methanol reactor 320 may then be directed to the methanol purification unit 330 to separate the methanol product stream 331 from other constituents 336 of the methanol reactor effluent 322. As the low-carbon H₂ in the low-carbon H₂ feed stream 110 and the CO₂ in the captured CO₂ feed stream 120 represent sustainable feed streams, the methanol product stream 331 generated in the methanol synthesis section 300 includes both conventional methanol 334 and low-carbon methanol 332. At least a portion of the methanol product stream 331 may be certified as low-carbon methanol 332. The methanol product stream 331 may be divided into conventional methanol 334 and the low-carbon methanol 332, where the low-carbon methanol 332 refers to the portion of the methanol product stream 331 attributable to the introduction of the low-carbon H₂ feed stream 110, the captured CO₂ feed stream 120, or both to the methanol production unit 10.

The processes disclosed herein may include introducing the low-carbon H₂ feed stream 110, the captured CO₂ feed stream 120, or both to the methanol production unit 10 upstream of the methanol synthesis reactor 320 and downstream of the secondary reformer 230, such as downstream of the heat recovery unit 240. In embodiments, the processes may include introducing the low-carbon H₂ feed stream 110, the captured CO₂ feed stream 120, or both to the methanol production unit 10 downstream of the secondary reformer 230 and upstream of the syngas compression unit 310, such as downstream of the heat recovery unit 240 and upstream of the syngas compression unit 310. In embodiments, the process may include combining the low-carbon H₂ feed stream 110, the captured CO₂ feed stream 120, or both with the syngas stream 250 downstream of the secondary reformer 230 to produce a combined syngas stream 130; compressing the combined syngas stream 130 in the syngas compression unit 310 to produce a pressurized combined syngas stream; and passing the compressed combined syngas stream to the methanol synthesis reactor 320.

In embodiments, the processes may include introducing the low-carbon H₂ feed stream 110, the captured CO₂ feed stream 120, or both to the methanol production unit 10 downstream of the syngas compression unit 310 and upstream of the methanol reactor 320, such as combining the low-carbon H₂ feed stream 110, the captured CO₂ feed stream 120, or both with the pressurized syngas stream 312 upstream of the methanol reactor 320 and downstream of the syngas compression unit 310.

The relative amounts of the low-carbon H₂ feed stream 110 and the captured CO₂ feed stream 120 may be determined based on the desired composition of the compressed syngas stream 312 introduced to the methanol reactor 320. A flow rate of the low-carbon H₂ feed stream 110 and a flow rate of the captured CO₂ feed stream 120 may be sufficient so that the syngas molar ratio (i.e., [(moles H₂)−(moles CO₂)]/[(moles CO)+(moles CO₂)]) of the compressed syngas stream 312 passed to the methanol reactor 320 is from 1.8 to 2.4, from 1.8 to 2.2, from 1.9 to 2.4, from 1.9 to 2.2, from 2.0 to 2.4, or from 2.0 to 2.2. In embodiments, the flow rate of the low-carbon H₂ feed stream 110 and the flow rate of the captured CO₂ feed stream 120 may be sufficient so that the syngas molar ratio is stoichiometric in the compressed syngas stream 312 passed to the methanol reactor 320.

In embodiments, the low-carbon H₂ feed stream 110 and the captured CO₂ feed stream 120 may be introduced to the methanol production unit 10 at a molar ratio of the low-carbon H₂ to the captured CO₂ of from 2 to 5, from 2 to 4, from 2 to 3.5, from 2.5 to 5, from 2.5 to 4, from 2.5 to 3.5, from 3 to 5, from 3 to 4, from 3 to 3.5, or from 3.5 to 5. As discussed in this paragraph, the molar ratio of low-carbon H₂ to captured CO₂ is equal to the molar flow rate of low-carbon H₂ from the low-carbon H₂ feed stream 110 divided by the molar flow rate of captured CO₂ from the captured CO₂ feed stream 120. The molar ratio of low-carbon H₂ to captured CO₂ in this paragraph does not include any of the H₂ and CO₂ in the primary reformer outlet stream 224 or syngas stream 250 that was present in the natural gas stream 102 or derived from constituents in the natural gas stream 102.

In embodiments, the low-carbon H₂ feed stream 110 and the captured CO₂ feed stream 120 may be introduced to the methanol production unit 10 at molar flow rates resulting in a mole percentage of the low-carbon feed components of from 0.01 mol % to 40 mol %, where the weight percentage of the low-carbon feed components is equal to the sum of the molar flow rates of the low-carbon H₂ feed stream 110 and the captured CO₂ feeds stream 120 divided by the total molar flow rate of the pressurized syngas stream 312 introduced to the methanol reactor 320. In embodiments, the mole percentage of the low-carbon feed components (low-carbon H₂ feed stream 110, the captured CO₂ feeds stream 120, or both) introduced to the methanol production unit 10 may be from 0.01 mol % to 30 mol %, from 0.01 mol % to 20 mol %, from 0.01 mol % to 10 mol %, from 0.01 mol % to 5 mol %, from 0.1 mol % to 40 mol %, from 0.1 mol % to 30 mol %, from 0.1 mol % to 20 mol %, from 0.1 mol % to 10 mol %, from 0.1 mol % to 5 mol %, from 1 mol % to 40 mol %, from 1 mol % to 30 mol %, from 1 mol % to 20 mol %, from 1 mol % to 10 mol %, from 1 mol % to 5 mol %, from 5 mol % to 40 mol %, from 5 mol % to 30 mol %, from 5 mol % to 20 mol %, or from 5 mol % to 10 mol %.

In embodiments, the captured CO₂ feed stream 120 may be introduced to the methanol production unit 10, and a weight ratio of the natural gas stream 102 to the captured CO₂ feed stream 120 introduced to the methanol production unit 10 may be from 0.01 to 40, such as from 0.1 to 30, from 1 to 20, from 10 to 15, or about 14.4. In embodiments, the low-carbon H₂ feed stream 110 may be introduced to the methanol production unit 10, and a weight ratio of the natural gas stream 102 to the low-carbon H₂ feed stream 110 introduced to the methanol production unit 10 may be from 1 to 200, such as from 10 to 150, from 50 to 150, from 75 to 125, or about 105.

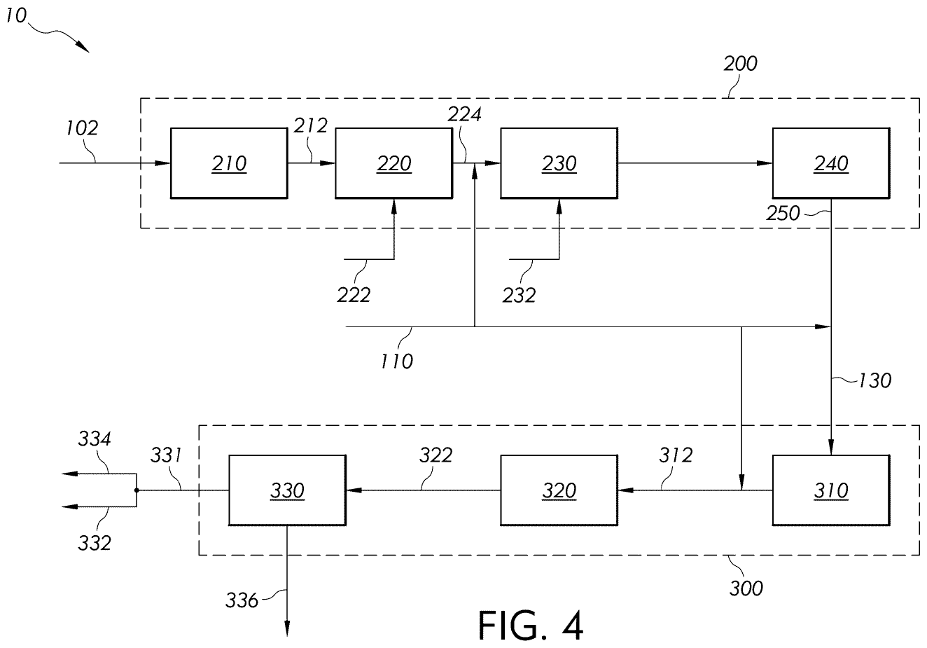

Referring now to FIG. 4, in embodiments, the low-carbon H₂ in the low-carbon H₂ feed stream 110 may be combined with the syngas stream 250 produced according to any scheme within the existing methanol production unit 10. It is noted that in accordance with the embodiments in FIG. 4, no captured CO₂ feed stream 120 is provided to the methanol production unit 10. As previously discussed, the low-carbon H₂ feed stream 110 may include green H₂, blue H₂, pink H₂, turquoise H₂, reduced carbon sustainable H₂, H₂ produced from H₂S splitting, or combinations of these. In embodiments, the low-carbon H₂ feed stream 110 may comprise, consist of, or consist essentially of blue H₂, pink H₂, turquoise H₂, H₂ produced from flue gas or waste gas with low-carbon emissions, H₂ produced from H₂S splitting, or any combinations thereof. In embodiments, the low-carbon H₂ feed stream 110 is selected from the group consisting of blue H₂, pink H₂, red H₂, purple H₂, turquoise H₂, H₂ produced from flue gas or waste gas with low-carbon emissions, H₂ produced from H₂S splitting, and combinations thereof.

The low-carbon H₂ in the low-carbon H₂ feed stream 110 may be combined with the syngas stream 250 upstream of the syngas compression unit 310 and downstream of the secondary reformer 230. In embodiments, the low-carbon H₂ in the low-carbon H₂ feed stream 110 may be combined with the syngas stream 250 downstream of the heat recovery unit 240 and upstream of the syngas compression unit 310. The combined stream 130 formed from the syngas stream 250 and the low-carbon H₂ feed stream 110 may then be fed to the methanol synthesis section 300 of the existing methanol production unit 10. In embodiments, the combined stream 130 may be fed into the syngas compression unit 310 to be pressurized to produce the pressurized syngas stream 312, which may be passed to the methanol reactor 320 to produce methanol. In embodiments, the low-carbon H₂ feed stream 110 may be combined with the pressurized syngas stream 312 downstream of the syngas compression unit 310 and upstream of the methanol reactor 320 to produce a pressurized combined stream.

The methanol reactor 320 may convert constituents of the syngas stream 250 and low-carbon H₂ feed stream 110 through one or more of Reactions 1-3 to produce the methanol reactor effluent 322 comprising methanol. The methanol reactor effluent 322 from the methanol reactor 320 may be passed to the methanol purification unit 330 to separate methanol from other products to produce the methanol product stream 331 and other constituents 336. Since the low-carbon H₂ in the low-carbon H₂ feed stream 110 represents a sustainable feed, the methanol product stream 331 generated in the methanol synthesis section 300 includes both conventional methanol 334 and low-carbon methanol 332, which can be e-methanol.

Referring again to FIG. 4, in embodiments, the low-carbon H₂ in the low-carbon H₂ feed stream 110 may be may be fed into a reformer in the syngas synthesis section 200, such as the primary reformer 220, the secondary reformer 230, or both. In embodiments, no captured CO₂ feed stream 120 is provided to the methanol production unit 10. The low-carbon H₂ feed stream 110 may be introduced directly to the secondary reformer 230 or combined with the primary reformer outlet stream 224 downstream of the primary reformer 220 and upstream of the secondary reformer 230. Introducing the low-carbon H₂ feed stream 110 into the secondary reformer 230 produces CO and H₂O through reaction of the H₂ from the low-carbon H₂ feed stream 110 and CO₂ from processing of the natural gas stream 102. Accordingly, the composition of the syngas stream 250 may be controlled. The syngas stream 250 may then be processed in the methanol synthesis section 300 to generate methanol. As the low-carbon H₂ in the low-carbon H₂ feed stream 110 represents a sustainable feed which was utilized to produce a portion of the syngas stream 250 fed to the methanol synthesis section 300, the methanol generated in the methanol synthesis section 300 includes both conventional methanol 334 and low-carbon methanol 332, such as e-methanol.

Referring again to FIG. 4, in embodiments, the low-carbon H₂ feed stream 110 may be introduced to the methanol production unit 10 in multiple locations. In embodiments, a first portion of the low-carbon H₂ feed stream 110 may be combined with the primary reformer outlet stream 224 upstream of the secondary reformer 230, and a second portion of the low-carbon H₂ feed stream 110 may be combined with the syngas stream 250 downstream of the secondary reformer 230 and upstream of the methanol reactor 320, either before or after the syngas compression unit 310.

Referring again to FIGS. 2-4, as previously discussed, the methanol reactor 320 may produce the methanol reactor effluent 322, which may be separated in the methanol purification unit 330 to produce a methanol product stream 331 and a stream containing other constituents 336. The methanol product stream 331 may include low-carbon methanol 332 and conventional methanol 334. The low-carbon methanol 332 is the portion of the total methanol that is attributable to introduction of the low-carbon H₂ feed stream 110, the captured CO₂ feed stream 120, or both to the existing methanol production unit 10. The conventional methanol 334 is the rest of the methanol presumed to be derived from the natural gas stream 102.

It will be appreciated that the low-carbon methanol 332 and the conventional methanol 334 in the methanol product stream 331 are intermingled. That is, the methanol product stream 331 passed out of the methanol purification unit 330 includes a combination of conventional methanol and low-carbon certifiable methanol. Accordingly, the molecules of conventional methanol and the molecules of low-carbon methanol are indistinguishable and may not be practically separated. However, the quantity of the low-carbon methanol can be determined and certified using a mass balance method, an energy balance method, an atom-based tracing method, or other certification method, as further described in the present disclosure. The low-carbon methanol 332 is the portion of the total methanol in the methanol product stream 331 that is attributed to introducing the captured CO₂ feed stream 120, the low-carbon H₂ feed stream 110, or both to the methanol production unit 10 based on a mass balance certification basis, an energy balance certification basis, a trace-the-atom certification basis, or other certification basis. In embodiments, the low-carbon H₂ feed stream 110 may be green H₂ and the low-carbon methanol 332 may be certified as e-methanol on a mass basis, energy basis, trace-the-atom basis, or other basis. E-methanol refers to methanol that meets the standards for e-chemicals in RED II. The quantity of low-carbon methanol or e-methanol produced may be certified using the guidelines established under the ISCC EU, the ISCC, or certification programs under another such equivalent certification body. In embodiments, the processes disclosed herein may include certifying a portion of the methanol in the methanol product stream 331 as low-carbon methanol using the mass balance certification basis, the energy balance certification basis, the trace-the-atoms certification basis, other certification basis, or combinations thereof. In embodiments, a proportion of the low-carbon methanol 332 in the methanol product stream 331 may be from 0.01 weight percent (wt. %) to about 40 wt. % based on the total weight of methanol in the methanol product stream 331.

It will be appreciated that one or more of the operating parameters of the methanol production unit 10 may be adjusted to account for changes in reactor compositions with the feeding of sustainable feedstock into the process. These changes may include, but are not limited to, changes in temperature, pressure, LHSV, catalyst compositions, and ancillary stream flow rates (such as steam or oxygen flow rates). For example, the temperature of a reactor may be adjusted in response to the change in stream compositions from the provision of low-carbon H₂ and captured CO₂ in order to influence the yield and selectivity of methanol in the methanol production unit 10.

### Low-Carbon Fuels and Chemicals Production

Low-carbon fuels and chemicals may be produced from the low-carbon methanol 332 or other low-carbon oxygenate streams using existing hydrocarbon process schemes. Generation of low-carbon fuels and chemicals may be in accordance with various process schemes to allow for utilization of existing production units. Specifically, fluid catalytic cracking (FCC) is commonly used to generate gasoline, olefins, and other fuels and chemicals from conventional FCC feeds, such as but not limited to vacuum gas oil (VGO), atmospheric residue (AR), or both. Fluid catalytic cracking (FCC) is a process that converts heavy petroleum fractions into lighter hydrocarbon products. FCC processes are common operations in oil and gas refineries around the world, and one having skill in the art has familiarity with FCC units and their operation. In the FCC process, heavy hydrocarbon fractions, such as heavy gas oils, vacuum gas oils, atmospheric residues, or other heavy fractions are heated to operating temperatures greater than or equal to 315° C. (600° F.), such as from 315° C. to 800° C., from 400° C. to 700° C., or from 480° C. to 550° C., and then mixed with a catalyst in a fluidized catalytic cracking reactor. The catalyst and heat break apart the larger hydrocarbon molecules into smaller hydrocarbon molecules, such as those that make up gasoline, middle distillates, olefins, and other greater-value products. A low-carbon oxygenate stream, such as the low-carbon methanol 332 produced from the methanol production unit 10, can be co-fed with a conventional FCC feed to an existing FCC unit to produce fuels and chemicals, at least a portion of which can be certified as low-carbon fuels and chemical based on a mass certification basis, an energy certification basis, a trace-the-atom certification basis, other certification basis, or combinations thereof.

Referring now to FIG. 5, an existing FCC unit 400 for upgrading a conventional FCC feed 405 to produce fuels and chemicals is schematically depicted. The FCC unit 400 may include an FCC reactor 410, a catalyst separation section 413 at a downstream end of the FCC reactor 410, an FCC effluent separation unit 420, and a catalyst regenerator 430.

The FCC reactor 410 may be a riser reactor, as shown in FIG. 5. In embodiments, the FCC reactor 410 may be a downer reactor. The conventional FCC feed 405 may be introduced to the inlet end 411 of the FCC reactor 410 along with an FCC catalyst, such as the regenerated FCC catalyst 434. In embodiments, the FCC catalyst may include fresh FCC catalyst in addition to or in place of the regenerated FCC catalyst 434. In embodiments, a fluidization gas 406 may be introduced to the inlet end 411 of the FCC reactor 410 to facilitate fluidization of the FCC catalyst. The FCC catalyst and the conventional FCC feed 405 may intermix at the inlet end 411 of the FCC reactor 410 and may flow together through the FCC reactor 410 from the inlet end 411 to the outlet end 412 of the FCC reactor 410.

The FCC reactor 410 may be operated at a temperature sufficient to cause catalytic cracking of hydrocarbons from the conventional FCC feed 405 to produce fuels and chemicals. In embodiments, the FCC reactor 410 may be operated at a temperature of from 315° C. to 800° C., such as from 400° C. to 750° C., from 400° C. to 700° C., from 450° C. to 700° C., from 500° C. to 650° C., from 480° C. to 550° C., or any range or subrange therein. A weight ratio of the FCC catalyst to the hydrocarbons in the FCC reactor 410 (the catalyst to hydrocarbon ratio) may be from 2:1 to 40:1, from 5:1 to 40:1, from 10:1 to 35:1, from 15:1 to 30:1, from 15:1 to 25:1, or any range or subrange therein. A residence time of the hydrocarbons with the FCC catalyst in the FCC reactor 410 at the reaction temperature may be from 0.2 seconds (sec) to 30 sec, or from 0.2 seconds to 3 seconds.

Contact of the hydrocarbons in the conventional FCC feed 405 with the FCC catalyst at the reaction conditions in the FCC reactor 410 may cause catalytic cracking of some of the hydrocarbons to upgrade the hydrocarbons to produce greater value fuels, chemicals, or both. Fuels may refer to hydrocarbons that are useful as fuel blending components. Chemicals may refer to chemical intermediates having value as intermediates in the production of other chemical products or plastics. Chemicals may include but are not limited to light olefins (C2-C4 olefins), light aromatic compounds (C6-C8 aromatic compounds such as but not limited to benzene, toluene, xylenes, or others), or other chemicals, or combinations thereof. In embodiments, the greater value fuels, chemicals, or both may include fuel gas (methane), light petroleum gases (propane, butane, isobutane, etc.), light olefins (propylene, ethylene, mixed butenes), light aromatic compounds (benzene, toluene, xylenes, ethylbenzene, or other C6-C8 aromatic compounds), gasoline (boiling point temperatures of from 30° C. to 225° C.), cycle oils (boiling point temperature range of from 225° C. to 400° C.), or combinations thereof.

Referring again to FIG. 5, the reaction mixture comprising the used FCC catalyst, reaction products, unreacted hydrocarbons from the conventional FCC feed 405, and optionally any carrier gases, pass from the FCC reactor 410 to the catalyst separation section 413 disposed downstream of the FCC reactor 410. The catalyst separation section 413 operates to separate the reaction mixture into an FCC effluent 414 and used FCC catalyst 416. The catalyst separation section 413 may be disposed at the outlet end 412 of the FCC reactor 410 and may include one or a plurality of solid fluid separation devices.

Referring again to FIG. 5, the used FCC catalyst 416 may be passed to the catalyst regenerator 430, which may be operable to regenerate the used FCC catalyst 416 to produce the regenerated catalyst 434. The processes disclosed herein may include regenerating the used FCC catalyst 416 in the catalyst regenerator 430 to produce the regenerated FCC catalyst 434. The used FCC catalyst 416 may be regenerated in the catalyst regenerator 430 according to any method and system known to those skilled in the art. Regenerating the used FCC catalyst 416 may include removing coke deposits from the used FCC catalyst 416, heating the used FCC catalyst 416 to a temperature greater than or equal to the operating temperature of the FCC reactor 410, or combinations thereof. Removing coke deposits and/or heating the used FCC catalyst 416 to produce the regenerated FCC catalyst 434 may include introducing regeneration gases 432 to the regenerator 430. The regeneration gases 432 may include an oxygen-containing gas and optionally a fuel gas. Combustion of the coke deposits, the fuel gas, or both in the presence of the oxygen-containing gas may remove the coke deposits from the FCC catalyst and heat the FCC catalyst to produce the regenerated FCC catalyst 434 and a flue gas 436. The regenerated FCC catalyst 434 may be passed back to the inlet end 411 of the FCC reactor 410 and the flue gas 436 may be passed out of the regenerator 430.

The FCC catalyst may include any known or future developed FCC catalyst suitable for use in the FCC reactor 410. The catalyst may be a heat carrier and may provide heat transfer to the hydrocarbons in the FCC reactor 410. The catalyst may also have a plurality of catalytically active sites, such as acidic sites for example, that promote cracking reactions. Examples of FCC catalysts suitable for use in the FCC reactor 410 may include, without limitation, zeolites, silica-alumina catalysts, carbon monoxide burning promoter additives, bottoms cracking additives, light olefin-producing additives, other catalyst additives, or combinations of these components. Zeolites may include, but are not limited to, Y zeolite, REY zeolites, USY zeolites, RE-USY zeolites, beta zeolites, or combinations of these. The FCC catalyst may also include a shape-selective catalyst additive, such as but not limited to ZSM-5 zeolites or other pentasil-type catalyst structures, which are often used in FCC processes to produce light olefins and/or increase FCC gasoline octane. In embodiments, the FCC catalyst may include one or more zeolite components in combination with clays, alumina, binders, or combinations of these. In embodiments, the FCC catalysts may be formed into pellets through extrusion, calcining, and sizing.

In embodiments, at least a portion of the catalyst may be modified to include one or more rare earth elements (15 elements of the Lanthanide series of the IUPAC Periodic Table plus scandium and yttrium), alkaline earth metals (Group 2 of the IUPAC Periodic Table), transition metals, phosphorus, fluorine, or any combination of these, which may influence yields of one or more constituents in the FCC effluent 414. One or more transition metals or metal oxides may also be impregnated onto the catalyst. Metals or metal oxides may include one or more metals from Groups 6-10 of the IUPAC Periodic Table. In embodiments, the metals or metal oxides may include one or more of molybdenum, rhenium, tungsten, or any combination of these. Any other FCC catalysts suitable for use in an FCC reactor are contemplated. In embodiments, the FCC catalyst may include a primary FCC catalyst and an FCC additive, such as a cracking additive included to promote selectivity of the cracking reactions to one or more specific products or groups of products.

Referring again to FIG. 5, the FCC effluent 414 may be passed from the catalyst separation section 413 to the FCC effluent separation unit 420. The FCC effluent separation unit 420 may include one or a plurality of separation units operable to separate the FCC effluent 414 into one or more product streams 421 comprising the greater value fuels and chemicals. The FCC effluent separation unit 420 may be operable to further produce a light gas stream 426, a bottom stream 428, or both. The light gas stream 426 may include methane, ethane, hydrogen, or other light gases produced in the FCC reactor 410 and may be a fuel gas stream, which can be combusted to produce heat in one or more other refinery systems. The bottom stream 428 may include heavier boiling constituents of the FCC effluent 414 (constituents having boiling point temperatures greater than 400° C.), which may be further processed or recycled back to the FCC reactor 410 for further processing in the FCC unit 400. The processes disclosed herein may include separating the FCC effluent 414 in the FCC effluent separation unit 420 to produce at least one product stream 421 comprising fuels, chemicals, or both. The at least one product stream 421 may include an LPG stream, a light olefin stream, a gasoline stream, a cycle oil stream, or any combinations thereof.

Referring again to FIG. 5, the conventional FCC feed 405 may include crude oil, vacuum residue, tar sands, bitumen, atmospheric residue, vacuum gas oils, demetalized oils, naphtha streams, other hydrocarbon streams, or combinations of these materials. In embodiments, the conventional FCC feed 405 may include vacuum gas oils, atmospheric residue, vacuum residue, or combinations of these. In embodiments, the conventional FCC feed 405 may be a crude oil. As previously discussed, the conventional FCC feed 405 may be introduced to the FCC reactor 410, such as by being injected into the FCC reactor 410, at the inlet end 411 of the FCC reactor 410.

As previously discussed, the existing FCC unit 400 may be leveraged to produce low-carbon fuels and chemicals. For the production of sustainable low-carbon fuels and chemicals, such as low-carbon gasoline, low-carbon light olefins, or both, low-carbon oxygenates may be co-fed with conventional FCC feed to the FCC reactor. The arrangement of the co-feeding may be provided in different ways based on the existing FCC plant configuration.

Referring again to FIG. 5, in embodiments, a low-carbon oxygenate stream 402 may be co-fed to the FCC reactor 410 along with the conventional FCC feed 405. In embodiments, the low-carbon oxygenate stream 402 may include the low-carbon methanol 332 produced from the methanol production unit 10 through introduction of the low-carbon H₂ feed stream 110, the captured CO₂ stream 120, or both to the methanol production unit 10. The low-carbon oxygenate stream 402 may include low-carbon oxygenates produced from other sources, such as but not limited to biogenic oxygenates. As used herein, the term “biogenic oxygenates” refer to oxygenates produced from biological materials, such as plant matter or waste organic matter. In embodiments, the biogenic oxygenates can include but are not limited to bio-methanol, bio-ethanol, bio-dimethyl ether, other bio oxygenate, or combinations of these bio-oxygenates. In embodiments, the low-carbon oxygenate stream 402 may include a combination of the low-carbon methanol 332 and one or more biogenic oxygenate streams. The processes disclosed herein may include co-feeding of biogenic oxygenates, such as but not limited to bio-methanol, bio-ethanol, bio-dimethyl ether, or combinations of these, with the conventional FCC feeds 405 in the FCC unit 400 to produce low-carbon fuels or olefins.

In embodiments, the low-carbon oxygenate stream 402 may be combined with the conventional FCC feed 405 upstream of the FCC reactor 410, and the combined stream may be injected into the FCC reactor 410. In embodiments, the low-carbon oxygenate stream 402 may be injected into the FCC reactor 410 through a different set of nozzles and at a different point within the FCC reactor 410 compared to introduction of the conventional FCC feed 405 into the FCC reactor 410. In embodiments, the low-carbon oxygenate stream 402 may be introduced to the FCC reactor 410 at a different elevation within the FCC reactor 410 compared to the conventional FCC feed 405. In general, the low-carbon oxygenates in the low-carbon oxygenate stream 402 may be easier to crack compared to the heavier hydrocarbons in the conventional FCC feed 405 and may require a shorter residence time in the FCC reactor 410. Therefore, the low-carbon oxygenate stream 402 may injected into the FCC reactor 410 at an injection point upstream from the injection point of the conventional FCC feed 405. The positioning and flow rate from each set of nozzles may be selected based on the composition of each of the low-carbon oxygenate stream 402 and the conventional FCC feed 405. In embodiments, the low-carbon oxygenate stream 402 may be a biogenic oxygenate stream comprising bio-methanol, bio-ethanol, bio-dimethyl ether, or combinations thereof. The biogenic oxygenate stream can be fed to the existing FCC unit 400 with the conventional FCC feed 405 or may be injected through a separate set of nozzles at a certain elevation of the FCC reactor 410.

The products generated in the FCC reactor 410 from the combination of the feed of low-carbon oxygenate stream 402 and the conventional FCC feed 405 are present in the FCC reactor effluent 414 passed out of the catalyst separation section 413. As previously discussed, the FCC effluent 414 can be separated in the existing FCC effluent separation system 420 to produce at least one product stream 421. As the feed of low-carbon oxygenate stream 402 represents a sustainable feed, the fuels and chemicals produced in the FCC reactor 410 and present in the at least one product stream 421 includes both conventional fuels and chemicals 424 and low-carbon fuels and chemicals 422. In embodiments, the low-carbon fuels and chemicals 422 may include e-fuels, e-olefins, or combinations of both.

Referring again to FIG. 5, the existing FCC unit 400 may be used in a process for producing sustainable or low-carbon fuels and chemicals. The processes may include catalytically cracking the conventional FCC feed 405 with an FCC catalyst in the FCC reactor 410 to produce the FCC effluent 414 comprising fuel and chemical components. The processes may further include injecting a low carbon oxygenate stream, such as the low-carbon oxygenate stream 402, into the FCC reactor 410, wherein the low-carbon oxygenate stream comprises oxygenate compounds produced from sustainable sources using captured CO₂, low-carbon hydrogen sources, biogenic sources, or combinations thereof. The processes may include recovering the FCC effluent 414 and certifying at least a portion of the FCC effluent 414 as the low-carbon fuels and chemical 422, wherein the low-carbon fuels and chemicals 422 are the fuel and chemical components attributed to injection of the low-carbon oxygenate stream 402 to the FCC reactor 410 based on a mass balance certification basis, an energy balance certification basis, a trace-the-atom certification basis, or other certification basis.

In embodiments, the low-carbon oxygenate stream 402 may include low-carbon methanol. In embodiments, the processes may include producing the low-carbon methanol 332 according to any of the processes described in the present disclosure. The processes may include injecting the low-carbon methanol 332 directly into the FCC reactor 410 upstream of the injection point of the conventional FCC feed 405. In embodiments, the processes may include combining the low-carbon methanol 332 with other low-carbon or sustainable oxygenates to produce the low-carbon oxygenate stream 402.

Referring now to FIG. 6, in embodiments, the existing FCC unit may be a dual FCC reactor unit 500, which may include a first FCC reactor 510 and a second FCC reactor 540. The conventional FCC feed 405 and the low-carbon oxygenate stream 402 may be cracked in separated fluidized bed reactors. In embodiments, the conventional FCC feed 405 may be subjected to fluidized catalytic cracking in the first FCC reactor 510 and the low-carbon oxygenate stream 402 may be subjected to fluidized catalytic cracking in the second FCC reactor 540.

The dual FCC reactor unit 500 may include the first FCC reactor 510, a first catalyst separation section 520 disposed at an outlet of the first FCC reactor 510, and a first FCC catalyst regenerator 530. The conventional FCC feed 405 may be introduced to the first FCC reactor 510 and contacted with a first FCC catalyst, such as regenerated first FCC catalyst 532, at reaction conditions in the first FCC reactor, which may cause at least a portion of the hydrocarbons in the conventional FCC feed 405 to under catalytic cracking to produce conventional fuels and chemicals. The first FCC catalyst may be any of the FCC catalysts previously discussed in the present application. The first catalyst separation section 520 may separate the reaction mixture from the first FCC reactor 510 to produce the first FCC effluent 522 and the used first FCC catalyst 524. The used first FCC catalyst 524 may be regenerated in the first FCC catalyst regenerator 530 to produce the regenerated first FCC catalyst 532, which is passed back to the first FCC reactor 510.

The dual FCC reactor unit 500 may include the second FCC reactor 540, a second catalyst separation section 550 disposed at an outlet of the second FCC reactor 540, and a second FCC catalyst regenerator 560. The low-carbon oxygenate stream 402, which may include the low-carbon methanol 332, may be introduced to the second FCC reactor 540 and contacted with a second FCC catalyst, such as regenerated second FCC catalyst 562, at reaction conditions in the second FCC reactor 540, which may cause at least a portion of the low-carbon oxygenate stream 402 to under catalytic cracking to produce low-carbon fuels and chemicals. The second catalyst separation section 550 may separate the reaction mixture from the second FCC reactor 540 to produce the second FCC effluent 552 and the used second FCC catalyst 554. The used second FCC catalyst 554 may be regenerated in the second FCC catalyst regenerator 560 to produce the regenerated second FCC catalyst 562, which is passed back to the second FCC reactor 540.

The second FCC catalyst may be any FCC catalyst described in the present disclosure and may be the same or different from the first FCC catalyst. The first FCC catalyst may include a first primary FCC catalyst and a first FCC additive, and the second FCC catalyst may comprise a second primary FCC catalyst and a second FCC additive. In embodiments, the first primary FCC catalyst may be the same or different from the second primary FCC catalyst. In embodiments, the first FCC additive may be the same or different from the second FCC additive.

In embodiments, the low-carbon oxygenate stream 402 may be passed by itself to the second FCC reactor 540. In embodiments, a supplemental hydrocarbon stream 542 may also be injected into the second FCC reactor 540. The low-carbon oxygenate stream 402 may be injected into the second FCC reactor 540 at a different location than the supplemental hydrocarbon stream 542. In embodiments, the supplemental hydrocarbon stream 542 may be combined with the low-carbon oxygenate stream 402 upstream of the second FCC reactor 540 and injected as a combined stream into the second FCC reactor 540. In embodiments, the supplemental hydrocarbon stream 542 may be a portion of the conventional FCC feed 405. In embodiments, the supplemental hydrocarbon stream 542 may be a light hydrocarbon stream. Examples of light hydrocarbon streams suitable for the supplemental hydrocarbon stream 542 may include but are not limited to straight run naphtha, olefin-rich FCC light naphtha, coker light naphtha, or combinations of these. Heavier hydrocarbon streams may also be used for the supplemental hydrocarbon stream 542. In embodiments, the low-carbon oxygenate stream 402 may be a biogenic oxygenate stream comprising bio-methanol, bio-ethanol, bio-dimethyl ether, or combinations thereof. In embodiments, the biogenic oxygenate stream may also be injected into the second FCC reactor 540. The biogenic oxygenate stream may be injected into the second FCC reactor 540 at a different location than the supplemental hydrocarbon stream 542. In embodiments, the supplemental hydrocarbon stream 542 may be combined with the biogenic oxygenate stream 402 upstream of the second FCC reactor 540 and injected as a combined stream into the second FCC reactor 540.

The catalyst composition of the second fluid bed reactor 540 and the first fluid bed reactor 510 may be the same or different. In embodiments, the first FCC reactor 510 and the second FCC reactor 540 may utilize the same or substantially the same FCC catalyst. In embodiments, the second FCC catalyst in the second FCC reactor 540 may be a different type of FCC catalyst compared to the first FCC catalyst in the first FCC reactor 510. The separation of the first FCC reactor 510 and the second FCC reactor 540 within the dual FCC reactor unit 500 may allow for each FCC reactor to have catalyst and operating conditions configured and adjusted based on the different feeds to produce the desired products. Specifically, the second FCC reactor 540, which receives the low-carbon oxygenate stream 402, may have a catalyst and operating conditions selected to increase the yield of low-carbon fuels and low-carbon olefins from the second FCC reactor 540. Selection of specific catalysts is within the knowledge of one skilled in the art.

It will be appreciated that when separate catalyst compositions are utilized for the first FCC reactor 510 and the second FCC reactor 540, separate catalyst regenerators, such as first FCC catalyst regenerator 530 and second FCC catalyst regenerator 560, may be deployed to maintain separation of the different catalyst types. In embodiments, the first FCC catalyst in the first FCC reactor 510 and the second FCC catalyst in the second FCC reactor 540 may be the same, in which case a single or combined catalyst regenerator may be deployed to process catalyst from both the first FCC reactor 510 and the second FCC reactor 540. Utilization of a single regenerator dual catalyst (SRDC) allows for a single catalyst regenerator to be used to process an FCC catalyst which is formulated for utilization in both first FCC reactor 510 and the second FCC reactor 540.

Referring again to FIG. 6, the first FCC effluent 522 and the second FCC effluent 552 may be combined to produce a combined FCC effluent 570. The dual FCC reactor system 500 may further include the FCC effluent separation system 420. The combined FCC effluent 570 can be separated in the existing FCC effluent separation system 420 to produce at least one product stream 421. The FCC effluent separation system 420 may further separate the combined FCC effluent 570 to produce a light gas stream 426, a bottom stream 428, or both. As the feed of low-carbon oxygenate stream 402 represents a sustainable feed, the fuels and chemicals produced in the second FCC reactor 540 and present in the at least one product stream 421 includes both conventional fuels and chemicals 424 and low-carbon fuels and chemicals 422. In embodiments, the low-carbon fuels and chemicals 422 may include e-fuels, e-olefins, or combinations of both.

Referring again to FIG. 6, the dual FCC reactor system 500 may be used in a process for producing low-carbon fuels and chemicals. The processes may include producing the low-carbon oxygenate stream 402 comprising low-carbon oxygenate compounds; catalytically cracking the conventional FCC feed stream 405 with the first FCC catalyst in the first FCC reactor 510 to produce the first FCC effluent 522 and the used first FCC catalyst 524; and catalytically cracking the low-carbon oxygenate stream 402 with the second FCC catalyst in the second FCC reactor 540 to produce the second FCC effluent 552 and the used second FCC catalyst 554, wherein the second FCC reactor 540 is parallel to the first FCC reactor 510. The processes may further include passing the first FCC effluent 522 and the second FCC effluent 552 to the FCC effluent separation system 420 or combining the first FCC effluent 522 and the second FCC effluent 552 to produce a combined FCC effluent 570 and passing the combined FCC effluent 570 to the FCC effluent separation system 420. The processes may further include separating the first FCC effluent 522 and the second FCC effluent 552 in the FCC effluent separation system 420 to produce at least one product stream 421 comprising fuel and chemical components, and certifying at least a portion of the fuel and chemical components in the at least one product stream 421 as low-carbon fuel and chemical components, wherein the low-carbon fuel and chemical components are the fuel and chemical components attributed to fluidized catalytic cracking of the low-carbon oxygenate stream 402 in the second FCC reactor 540 based on a mass balance certification basis, an energy balance certification basis, or a trace-the-atom certification basis.

In embodiments, the low-carbon oxygenate stream 402 may include the low-carbon methanol 332 produced in the methanol production unit 10. In embodiments, producing the low-carbon oxygenate stream may comprise producing the low-carbon methanol according to any of the processes and methods disclosed in the present disclosure. In embodiments, catalytically cracking the low-carbon oxygenate stream 402 with the second FCC catalyst in the second FCC reactor 540 may include catalytically cracking a supplemental hydrocarbon stream 542 with the second FCC catalyst in the second FCC reactor 540 and injecting the low-carbon oxygenate stream 402 into the second FCC reactor 402 upstream of an injection point of the supplemental hydrocarbon stream 542 into the second FCC reactor 540.

Any existing FCC unit 400 or dual FCC reactor system 500 within an existing hydrocarbon production facility may be leveraged within the various embodiments of the processes and systems for generating low-carbon fuels and chemicals. However, for clarity and conciseness, example FCC unit 400 is illustrated in FIG. 5 and example dual FCC reactor system 500 is illustrated in FIG. 6. It will be appreciated that other styles of existing FCC units and configurations of the existing FCC units within a hydrocarbon production facility may be leveraged within the various embodiments by providing co-feeds in a substantially similar manner as described in relation to FIGS. 5 and 6.

It will be appreciated that one or more of the operating parameters of the FCC unit 400 or the dual FCC reactor system 500 may be adjusted to account for changes in reactor compositions with the feeding of the sustainable feedstock, such as the low-carbon oxygenate stream 402, low-carbon methanol 332, or both into the process. These changes may include, but are not limited to, changes in temperature, pressure, LHSV, catalyst compositions, and other co-fed stream flow rates (such as light hydrocarbon, VGO, or AR flow rates). For example, the temperature of a reactor may be modified in response to the change in stream compositions from the provision of low-carbon methanol and/or other low-carbon oxygenates.

### Low-Carbon Product Attribution

It will be appreciated that in accordance with the various embodiments, streams of conventional products and streams of low-carbon products are intermingled. That is the methanol product stream 331 passed out of the methanol purification unit 330 and the at least one product stream 421 passed out of the FCC effluent separation system 420 each include a combination of conventional constituents and low-carbon certifiable constituents (low-carbon methanol or low-carbon fuels and chemicals, respectively). Accordingly, the molecules of conventional methanol, fuels, olefins, chemicals, or other constituents and the molecules of low-carbon methanol, fuels, olefins, chemicals, or other constituents are indistinguishable and may not be practically separated. However, the quantity of the low-carbon methanol 332 in the methanol product stream 331 or the quantity of the low-carbon fuels and chemicals 422 in the at least one product stream 421 can be determined and certified using a mass balance method, an energy balance method, an atom-based tracing method, or other certification method.

In embodiments, the amount of e-methanol 332 (or other low-carbon products) produced can be determined using either a mass balance or energy balance attribution approach. It will be appreciated that other methods are also possible and the attribution method is not dispositive of the intended scope of the present disclosure. However, to provide an example of how the distribution of low-carbon products may be determined, a mass/energy attribution approach to quantify a low-carbon methanol portion of the methanol product is presented. The sustainable share is determined by calculating the mass/energy ratio of the sustainable feedstock over the overall feedstock, multiplied by the conversion factor (amount of feedstock that is converted into products, disregarding by-products like water). The actual values will depend on the actual operational data of the plant. It will be appreciated that depending on the applicability of the attribution method used, it may be possible to attribute all the sustainable share to one product over another, for example, to methanol rather than fuel gas, or vice versa, to fuel gas rather than methanol. Attributing the sustainable share to the products based on the sustainable share of the feed streams allows for selective attribution of low-carbon or sustainable status to products which are more desirable to be classified as such.

### EXAMPLES

Embodiments of systems and processes of the present disclosure will be further clarified by the following examples. The examples are illustrative in nature, and should not be understood to limit the subject matter of the present disclosure.

### Example 1: Attribution of Low-Carbon Methanol from a Methanol Production Unit

In Example 1, an example calculation is provided for attribution of low-carbon methanol from a methanol production unit, to which low-carbon H₂ and captured CO₂ are introduced upstream of the methanol reactor. The feed streams, the products produced, and the attribution calculations are provided in Tables 1-3. Specifically, in an example methanol production unit (i.e., production plant) with a combination of fossil-sourced feeds and sustainable feeds (e.g., captured CO₂, low-carbon H₂, or both), mass and energy flows are shown for the input and output streams in Tables 1 and 2. A conversion factor (CF) is first calculated on a mass or energy basis by quantifying the ratio of total mass/energy output of products (in this case, total mass flow rate of methanol and fuel gas produced, but not including water), divided by the total mass/energy input of the feed streams (total mass flow rate of the feed streams). For instance, the MCF (mass basis conversion factor) is calculated as the total mass flow rate of products produced (not including water) divided by the total mass flow rate of the feed streams input into the methanol production unit. The ECF (energy basis conversion factor) is calculated as the total energy of the products produced divided by the total energy of the feed streams. It is noted that for the calculation for ECF, the energy value of water is zero.

The sustainable share is then determined by the total input of mass or energy (energy is normalized by the methanol lower heating value (LHV)) by the CF. The sustainable share refers to the products produced that can be certified as low-carbon. For mass basis, the sustainable share can be determined by multiplying the total mass flow rate of the sustainable feed streams by MCF. For the energy basis calculation, the sustainable share can be estimated by multiplying the total rate of energy input by the ECF and then dividing by the LHV of the low-carbon methanol. The mass and energy flows will depend on the operating plant for the methanol production unit. In Example 1, 424 tons per day (TPD) of captured CO₂ and 58 TPD of low-carbon hydrogen are co-fed with 6100 TPD of syngas to produce a total of 5303 TPD of methanol, of which 388 TPD is certifiable as sustainable e-methanol using the mass attribution, or 239 TPD using the energy attribution. The calculations are shown in Table 3. The choice of attribution method may depend on the applicability of each attribution method to relevant regulations and/or certifications.

TABLE 1
Methanol Plant Feeds

| | | |
|---|---|---|
| Flow Rate | LHV | Energy Flow |
| (TPD) | (MJ/Kg) | (GJ/Day) |

Fossil Fuel Feeds

| | | | |
|---|---|---|---|
| Syngas | 6100 | 24 | 146400 |

Sustainable Feeds

| | | | |
|---|---|---|---|
| Captured CO₂ | 424 | 0 | 0 |
| Low-carbon H₂ | 58 | 120 | 6912 |
| Total | 482 | 14 | 6912 |

TABLE 2
Methanol Plant Products

| | | |
|---|---|---|
| Flow Rate | LHV | Energy Flow |
| (TPD) | (MJ/Kg) | (GJ/Day) |

Fossil Fuel Products

| | | | |
|---|---|---|---|
| Conventional Methanol | 5000 | 22 | 110000 |
| Conventional Fuel Gas | 0 | 19 | 0 |

Sustainable Products

| | | | |
|---|---|---|---|
| Low-Carbon Methanol | 303 | 22 | 6696 |
| Low Carbon Fuel Gas | 6 | 19 | 116 |
| H₂O | 173 | 0 | 0 |
| Total | 482 | 14 | 6786 |

TABLE 3
Conversion Factor and Sustainable Share Quantification

| | | |
|---|---|---|
| Mass Conversion | 0.806 | (Total output − water)/Total Input |
| Factor (MCF) | | [i.e. (5000 + 482 − 173)/(6100 + 482)] |
| Energy | 0.762 | Total energy output/Total Energy Input |
| Conversion | | [i.e. (110000 + 6786)/(146400 + 6912)] |
| Factor (ECF) | | |
| Mass Sustainable | 388 TPD | MCF * Total Sustainable Input |
| Share | | [i.e. 0.81 * 482] |
| Energy | 239 TPD | (ECF * Total Sustainable |
| Sustainable | | Input)/e-methanol LHV |
| Share | | ]i.e. (0.76 * 6912)/22] |

In Table 2, all of the fuel gas produced is attributed to the low-carbon fuel gas. This is because, the introduction of the low-carbon H₂ and captured CO₂ is what results in the formation of fuel gas, such as through the reactions between CO₂ and H₂. When only natural gas is used as the feed stream, no fuel gas is produced generally.

### Example 2: Attribution of Low-Carbon Fuels and Chemicals from an FCC Unit

In Example 2, an example calculation is provided for attribution of low-carbon fuels and chemicals from an FCC unit, to which the low-carbon products (low-carbon methanol and low-carbon fuel gas) of Example 1 is co-fed with vacuum gas oil to an FCC unit. In embodiments, the amount of low-carbon fuels and chemicals produced can be determined using either a mass balance or energy balance attribution approach. It will be appreciated that other methods are also possible and the attribution method is not dispositive of the intended scope of the present disclosure. However, to provide an example of how the distribution of low-carbon products may be determined, a mass/energy attribution approach to quantify a low-carbon fuels and chemicals portion of the products from the FCC unit is presented. The sustainable share is determined by calculating the mass/energy ratio of the sustainable feedstock over the overall feedstock, multiplied by the conversion factor (amount of feedstock that is converted into products, disregarding by-products like water). The actual values will depend on the actual operational data of the plant. It will be appreciated that depending on the applicability of the attribution method used, it may be possible to attribute all the sustainable share to one product over another, for example, to gasoline rather than olefins, or vice versa, to olefins rather than gasoline. Attributing the sustainable share to the products based on the sustainable share of the feed streams allows for selective attribution of low-carbon or sustainable status to products which are more desirable to be classified as such.

The calculations for attribution of products generated in a FCC unit of Example 2 are provided in Tables 4-6. Specifically, in an example FCC unit with low-carbon feeds (low-carbon methanol and fuel gas from Example 1) and conventional feeds (such as a vacuum gas oil (VGO) for example), mass and energy flows are shown for the input and output streams in Tables 4 and 5. The conversion factor (CF) is first calculated on mass basis (MCF) or on an energy basis (ECF), by quantifying the ratio of total mass/energy output of products (in this example, low-carbon gasoline, low-carbon LPG, etc.), divided by the total mass/energy input of the feeds. The sustainable share is then determined by multiplying the CF by the total input of mass or energy (energy is normalized by methanol lower heating value (LHV)). The mass and energy flows will depend on the operating plant. In this Example 2, 309 ton per day (TPD) of the low-carbon feed is co-fed with 6298 TPD of VGO to produce a total of 6608 TPD products, of which 138 TPD is certifiable as sustainable low-carbon gasoline using the energy attribution. The calculations are shown in Table 6. The choice of attribution method may depend on the applicability of each attribution method to relevant regulations and/or certifications.

TABLE 4
FCC Unit Feeds

| | | |
|---|---|---|
| Flow Rate | LHV | Energy Flow |
| (TPD) | (MJ/Kg) | (GJ/Day) |

Fossil Fuel Feeds

| | | | |
|---|---|---|---|
| VGO | 6298 | 44 | 277128 |

Sustainable Feeds

| | | | |
|---|---|---|---|
| Low-Carbon Feed | 309 | 20 | 6183 |
| Stream | | | |

TABLE 5
FCC Unit Products

| | Flow Rate | LHV | Energy Flow |
|---|---|---|---|
| | (TPD) | (MJ/Kg) | (GJ/Day) |
| Gasoline | 2614 | 43 | 112399 |
| LPG + C3 = | 899 | 46 | 41353 |
| Fuel Gas | 333 | 49 | 16336 |
| Cycle Oil/Bottoms | 2144 | 41.5 | 88962 |
| Water + CO + CO₂ | 172 | 0 | 0 |
| Coke | 446 | 30 | 13366 |
| Total | 6608 | | 272416 |

TABLE 6
Conversion Factor and Sustainable Share Quantification

| | | |
|---|---|---|
| Mass Conversion | 0.97 | (Total output − water)/Total Input |
| Factor (MCF) | | [i.e. (6608 − ~104)/(6298 + 309)] |
| Energy | 0.962 | Total energy output/Total Energy Input |
| Conversion | | [i.e. (272416)/(277128 + 6183)] |
| Factor (ECF) | | |
| Mass Sustainable | 129 TPD | MCF * e-Methanol |
| Share | (1077 bpd) | input − Water − CO − CO₂ |
| | | [i.e. 0.97 * 309 − 172] |
| Energy | 138 TPD | (ECF * Total Sustainable |
| Sustainable | (1153 bpd) | Input)/e-gasoline LHV |
| Share | | [i.e. (0.962 * 6183)/43] |

The low-carbon feed stream in Table 4 is the low-carbon methanol and low-carbon fuel gas from Table 2 (303 TPD low-carbon methanol+6 TPD low-carbon fuel gas).

It should now be understood the various aspects of the processes and systems for generating low carbon fuels and petrochemicals are described and such aspects may be utilized in conjunction with various other aspects.

A first aspect of the present disclosure may be directed to a process for producing low-carbon methanol, the process comprising upgrading a natural gas stream in a methanol production unit to produce a methanol reactor effluent, wherein the methanol production unit comprises a syngas synthesis section and a methanol synthesis section downstream of the syngas synthesis section; and introducing a captured CO₂ feed stream, a low-carbon hydrogen feed stream, or both to the syngas synthesis section or to a syngas stream downstream of the syngas synthesis section and upstream of a methanol reactor of the methanol synthesis section. At least a portion of the methanol reactor effluent comprises low-carbon methanol. The low-carbon methanol comprises a portion of total methanol in the methanol reactor effluent that is attributed to the introducing the captured CO₂ feed stream, the low-carbon hydrogen feed stream, or both to the methanol production unit based on a mass balance certification basis, an energy balance certification basis, or a trace-the-atom certification basis.

A second aspect of the present disclosure may include the first aspect, comprising introducing the captured CO₂ feed stream and the low-carbon hydrogen feed stream to the syngas synthesis section or to the syngas stream downstream of the syngas synthesis section and upstream of the methanol reactor of the methanol synthesis section.

A third aspect of the present disclosure may include the second aspect, wherein the low-carbon hydrogen feed stream and the captured CO₂ feed stream may be introduced to the methanol production unit at a molar ratio of low-carbon hydrogen to captured CO₂ of from 2 to 5.

A fourth aspect of the present disclosure may include any one of the first through third aspects, wherein a weight ratio of the natural gas stream to the low-carbon hydrogen feed stream introduced to the methanol production unit may be from 1 to 200.

A fifth aspect of the present disclosure may include any one of the first through fourth aspects, wherein a weight ratio of the natural gas stream to the captured CO₂ feed stream introduced to the methanol production unit is from 0.1 to 40.

A sixth aspect of the present disclosure may include any one of the first through fifth aspects, wherein the methanol production unit may be an existing methanol production unit.

A seventh aspect of the present disclosure may include any one of the first through sixth aspects, comprising introducing the captured CO₂ feed stream, the low-carbon hydrogen feed stream, or both to the syngas synthesis section of the methanol production unit.

An eighth aspect of the present disclosure may include any one of the first through seventh aspects, wherein the syngas synthesis section may comprise a primary reformer and a secondary reformer downstream of the primary reformer, and the process may comprise introducing the captured CO₂ feed stream, the low-carbon hydrogen feed stream, or both to the syngas synthesis section downstream of the primary reformer and upstream of the secondary reformer.

A ninth aspect of the present disclosure may include any one of the first through eighth aspects, wherein upgrading the natural gas stream in the methanol production unit to produce the methanol reactor effluent may comprise: hydrodesulfurizing the natural gas stream in a hydrodesulphurization unit to produce a desulphurized natural gas stream; reforming the desulphurized natural gas stream in the presence of steam in a primary reformer downstream of the hydrodesulphurization unit to produce a primary reformer outlet stream; reforming the primary reformer outlet stream in the presence of oxygen in a secondary reformer downstream of the primary reformer to produce a syngas stream comprising CO₂, CO, and H₂; and converting the syngas stream in a methanol reactor disposed downstream of the secondary reformer to produce the methanol reactor effluent.

A tenth aspect of the present disclosure may include the ninth aspect, comprising introducing the captured CO₂ feed stream, the low-carbon hydrogen feed stream, or both to the secondary reformer.

An eleventh aspect of the present disclosure may include the tenth aspect, comprising combining the captured CO₂ feed stream, the low-carbon hydrogen feed stream, or both with the primary reformer outlet stream upstream of the secondary reformer.

A twelfth aspect of the present disclosure may include any one of the ninth through eleventh aspects, wherein the primary reformer, the secondary reformer, or both may comprise one or more of a steam methane reforming unit, an autothermal reforming unit, a partial oxidation unit, a water gas shift reaction unit, or a combination thereof.

A thirteenth aspect of the present disclosure may include any one of the ninth through twelfth aspects, comprising introducing the captured CO₂ feed stream, the low-carbon hydrogen feed stream, or both to the methanol production section upstream of the methanol reactor.

A fourteenth aspect of the present disclosure may include any one of the ninth through thirteenth aspects, comprising introducing the captured CO₂ feed stream, the low-carbon hydrogen feed stream, or both to the methanol production unit downstream of the secondary reformer.

A fifteenth aspect of the present disclosure may include any one of the ninth through fourteenth aspects, further comprising compressing the syngas in a syngas compression unit disposed downstream of the secondary reformer and upstream of the methanol reactor to produce a pressurize syngas stream, and passing the pressurized syngas stream to the methanol reactor.

A sixteenth aspect of the present disclosure may include the fifteenth aspect, comprising introducing the captured CO₂ feed stream, the low-carbon hydrogen feed stream, or both to the methanol production plant downstream of the secondary reformer and upstream of the syngas compression unit.

A seventeenth aspect of the present disclosure may include any one of the ninth through sixteenth aspects, comprising: combining the captured CO₂ feed stream, the low-carbon hydrogen feed stream, or both with the syngas downstream of the secondary reformer to produce a combined syngas stream; compressing the combined syngas stream to produce a compressed combined syngas stream; and passing the compressed combined syngas stream to the methanol reactor.

An eighteenth aspect of the present disclosure may include any one of the ninth through seventeenth aspects, comprising passing only the low-carbon hydrogen feed stream to the methanol production unit downstream of the primary reformer and upstream of the secondary reformer, downstream of the secondary reformer and upstream of a syngas compression unit, downstream of the syngas compression unit and upstream of the methanol reactor, or combinations of these locations.

A nineteenth aspect of the present disclosure may include the eighteenth aspect, wherein a first portion of the low-carbon H₂ feed stream is combined with the primary reformer outlet stream upstream of the secondary reformer, and a second portion of the low-carbon H₂ feed stream is combined with the syngas stream downstream of the secondary reformer and upstream of the methanol reactor.

A twentieth aspect of the present disclosure may include either one of the eighteenth or nineteenth aspects, wherein no captured CO₂ is introduced to the methanol production unit.

A twenty-first aspect of the present disclosure may include any one of the first through twentieth aspects, further comprising purifying the methanol reactor effluent in a methanol purification unit downstream of the methanol reactor to produce a methanol stream comprising the low-carbon methanol and the conventional methanol.

A twenty-second aspect of the present disclosure may include any one of the first through twenty-first aspects, wherein a proportion of the low-carbon methanol in the methanol stream may be from 0.01 wt % to 40 wt % based on total mass of methanol in the methanol stream.

A twenty-third aspect of the present disclosure may include any one of the first through twenty-second aspects, wherein the low-carbon hydrogen feed stream may comprise one or more of the following: green hydrogen produced from water electrolysis; blue hydrogen produced from fossil fuels with carbon capture and sequestration; pink hydrogen produced from water electrolysis powered by nuclear energy; turquoise hydrogen produced from methane pyrolysis; hydrogen produced from a process of splitting of H₂S; hydrogen produced from flue gas or waste gas with low-carbon emissions; or combinations thereof.

A twenty-fourth aspect of the present disclosure may include any one of the first through twenty-third aspects, further comprising producing the low-carbon hydrogen feed stream.

A twenty-fifth aspect of the present disclosure may include the twenty-fourth aspect, wherein producing the low-carbon hydrogen feed stream may comprise one or more of the following: subjecting water to electrolysis using energy derived from wind, solar cells, nuclear power plant, or combinations thereof to produce hydrogen; converting fossil fuels to hydrogen and carbon compounds with carbon capture and sequestration; recovering hydrogen from a methane pyrolysis process; recovering hydrogen from a process of splitting of H₂S; recovering hydrogen from flue gas or waste gas with low-carbon emissions; or combinations thereof.

A twenty-sixth aspect of the present disclosure may include any one of the first through twenty-fifth aspects, wherein the captured CO₂ feed stream may comprise one or more of the following: CO₂ directly captured from the atmosphere; CO₂ produced from biogenic sources; CO₂ captured from industrial point sources; or combinations thereof.

A twenty-seventh aspect of the present disclosure may include any one of the first through twenty-sixth aspects, comprising certifying a portion of the methanol in the methanol reactor effluent as the low-carbon methanol using the mass balance certification basis, the energy balance certification basis, or the trace-the-atoms certification basis.

A twenty-eighth aspect of the present disclosure may include any one of the first through twenty-seventh aspects, and may be directed to a process of producing sustainable hydrocarbon fuels through fluidized catalytic cracking (FCC). The process may comprise: producing the low-carbon methanol according to the process of any one of the first through twenty-seventh aspects; catalytically cracking a conventional FCC feed with an FCC catalyst in an existing FCC reactor to produce an FCC effluent comprising fuel and chemical components; injecting the low-carbon methanol into the FCC reactor; recovering the FCC effluent; and certifying at least a portion of the FCC effluent as low-carbon fuel and chemical components, wherein the low-carbon fuel and chemical components are the fuel and chemical components attributed to injection of the low-carbon methanol to the FCC reactor based on a mass balance certification basis, an energy balance certification basis, or a trace-the-atom certification basis.

A twenty-ninth aspect of the present disclosure may include the twenty-eighth aspect, where injecting the low-carbon methanol into the FCC reactor may comprise combining the low-carbon methanol with the conventional FCC feed to produce a combined feed and injecting the combined feed into the FCC reactor; or injecting the low-carbon methanol into the FCC reactor separate from the conventional FCC feed.

A thirtieth aspect of the present disclosure may include either one of the twenty-eighth or twenty-ninth aspects, where the FCC catalyst may comprise a primary FCC catalyst and an FCC additive.

A thirty-first aspect of the present disclosure may include any one of the first through twenty-seventh aspects, and may be directed to a process of producing sustainable hydrocarbon fuels in a dual FCC reactor system. The process may comprise producing the low-carbon methanol according to the process of any one of the first through twenty-seventh aspects; catalytically cracking a conventional FCC feed with a first FCC catalyst in a first FCC reactor to produce a first FCC effluent and used first FCC catalyst; catalytically cracking the low-carbon methanol with a second FCC catalyst in a second FCC reactor to produce a second FCC effluent and a used second FCC catalyst, wherein the second FCC reactor is parallel to the first FCC reactor; passing the first FCC effluent and the second FCC effluent to an FCC effluent separation system; separating the first FCC effluent and the second FCC effluent in the FCC effluent separation system to produce at least on product stream comprising fuel and chemical components; and certifying at least a portion of the fuel and chemical components in the at least one product stream as low-carbon fuel and chemical components, wherein the low-carbon fuel and chemical components are the fuel and chemical components attributed to fluidized catalytic cracking of the low-carbon methanol in the second FCC reactor based on a mass balance certification basis, an energy balance certification basis, or a trace-the-atom certification basis.

A thirty-second aspect of the present disclosure may include the thirty-first aspect, wherein catalytically cracking the low-carbon methanol with the second FCC catalyst in the second FCC reactor may comprise: catalytically cracking a supplemental hydrocarbon stream with the second FCC catalyst in the second FCC reactor; and injecting the low-carbon methanol into the second FCC reactor upstream or downstream of an injection point of the supplemental hydrocarbon stream.

A thirty-third aspect of the present disclosure may include any one of the thirtieth through thirty-second aspects, where the first FCC catalyst comprises a first primary FCC catalyst and a first FCC additive, and the second FCC catalyst comprises a second primary FCC catalyst and a second FCC additive.

A thirty-fourth aspect of the present disclosure may include any one of the thirtieth through thirty-third aspects, wherein the first FCC catalyst may be the same as the second FCC catalyst.

A thirty-fifth aspect of the present disclosure may include any one of the first through twenty-seventh aspects, comprising introducing the low-carbon hydrogen feed stream to the syngas stream downstream of the syngas synthesis section and upstream of the methanol synthesis section.

A thirty-sixth aspect of the present disclosure may be directed to a process for producing sustainable fuel and chemical components, the process comprising upgrading a natural gas stream in a methanol production unit to produce a methanol reactor effluent, wherein the methanol production unit comprises a syngas synthesis section and a methanol synthesis section downstream of the syngas synthesis section; and introducing a low-carbon hydrogen feed stream to the methanol production unit upstream of a methanol reactor of the methanol synthesis section. A portion of total methanol in the methanol reactor effluent comprises low-carbon methanol. The low-carbon methanol comprises the portion of the total methanol that is attributed to the introducing the low-carbon hydrogen feed stream to the methanol production unit based on a mass balance certification basis, an energy balance certification basis, or a trace-the-atom certification basis. The process may further include passing the low-carbon methanol to an FCC unit; catalytically cracking a conventional FCC feed stream with an FCC catalyst in an FCC reactor to produce an FCC effluent comprising fuel and chemical components; injecting the low-carbon methanol into the FCC reactor; recovering the FCC effluent; and certifying at least a portion of the FCC effluent as low-carbon fuel and chemical components, wherein the low-carbon fuel and chemical components are the fuel and chemical components attributed to injection of the low-carbon methanol to the FCC reactor based on a mass balance certification basis, an energy balance certification basis, or a trace-the-atom certification basis.

A thirty-seventh aspect of the present disclosure may include the thirty-sixth aspect, comprising introducing the low-carbon hydrogen feed stream to a syngas stream downstream of the syngas synthesis section and upstream of the methanol synthesis section.

A thirty-eighth aspect of the present disclosure may include either one of the thirty-sixth or thirty-seventh aspects, wherein the methanol synthesis section may comprise a syngas compression unit and a methanol reactor disposed downstream of the syngas compression unit, and the process may comprise introducing the low-carbon hydrogen feed stream to the methanol synthesis section downstream of the syngas compression unit and upstream of the methanol reactor.

A thirty-ninth aspect of the present disclosure may include any one of the thirty-sixth through thirty-eighth aspects, wherein the syngas synthesis section may comprise a primary reformer and a secondary reformer downstream of the primary reformer, and the process may comprise introducing the low-carbon hydrogen feed stream to the syngas synthesis section downstream of the primary reformer and upstream of the secondary reformer, downstream of the secondary reformer, or both.

A fortieth aspect of the present disclosure may include the thirty-ninth aspect, wherein a first portion of the low-carbon H₂ feed stream may be combined with the primary reformer outlet stream upstream of the secondary reformer, and a second portion of the low-carbon H₂ feed stream may be combined with the syngas stream downstream of the secondary reformer and upstream of the methanol reactor.

A forty-first aspect of the present disclosure may include any one of the thirty-sixth through fortieth aspects, wherein no captured CO₂ is introduced to the methanol production unit.

A forty-second aspect of the present disclosure may be directed to a process for producing sustainable fuel and chemical components through fluidized catalytic cracking (FCC), the process comprising: catalytically cracking a conventional FCC feed with an FCC catalyst in an FCC reactor to produce an FCC effluent comprising fuel and chemical components; injecting a low-carbon oxygenate stream into the FCC reactor, wherein the low-carbon oxygenate stream comprises oxygenate compounds produced from sustainable sources using captured CO₂, low-carbon hydrogen sources, biologic sources, or combinations thereof; recovering the FCC effluent; and certifying at least a portion of the FCC effluent as low-carbon fuel and chemical components, wherein the low-carbon fuel and chemical components are the fuel and chemical components attributed to injection of the low-carbon oxygenate stream to the FCC reactor based on a mass balance certification basis, an energy balance certification basis, or a trace-the-atom certification basis.

A forty-third aspect of the present disclosure may be directed to a process for producing sustainable hydrocarbon fuels through fluidized catalytic cracking (FCC), the process comprising: producing a low-carbon oxygenate stream comprising low-carbon oxygenate compounds; catalytically cracking a conventional FCC feed stream with a first FCC catalyst in a first FCC reactor to produce a first FCC effluent and used first FCC catalyst; catalytically cracking the low-carbon oxygenate stream with a second FCC catalyst in a second FCC reactor to produce a second FCC effluent and a used second FCC catalyst, wherein the second FCC reactor is parallel to the first FCC reactor; passing the first FCC effluent and the second FCC effluent to an FCC effluent separation system; separating the first FCC effluent and the second FCC effluent in the FCC effluent separation system to produce at least one product stream comprising fuel and chemical components; and certifying at least a portion of the fuel and chemical components as low-carbon fuel and chemical components, wherein the low-carbon fuel and chemical components are the fuel and chemical components attributed to fluidized catalytic cracking of the low-carbon oxygenate stream in the second FCC reactor based on a mass balance certification basis, an energy balance certification basis, or a trace-the-atom certification basis.

A forty-fourth aspect of the present disclosure may include forty-third aspect, wherein catalytically cracking the low-carbon oxygenate stream with the second FCC catalyst in the second FCC reactor may comprise: catalytically cracking a supplemental hydrocarbon stream with the second FCC catalyst in the second FCC reactor; and injecting the low-carbon oxygenate stream into the second FCC reactor upstream or downstream of an injection point of the supplemental hydrocarbon stream.

A forty-fifth aspect of the present disclosure may include either one of the forty-third or forty-fourth aspects, where the first FCC catalyst may be the same as the second FCC catalyst.

It should be apparent to those skilled in the art that various modifications and variations can be made to the described embodiments without departing from the spirit and scope of the claimed subject matter. Thus, it is intended that the specification cover the modifications and variations of the various described embodiments provided such modifications and variations come within the scope of the appended claims and their equivalents.

It is noted that any two quantitative values assigned to a property may constitute a range of that property, and all combinations of ranges formed from all stated quantitative values of a given property are contemplated in this disclosure.

It is noted that one or more of the following claims utilize the term “where” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

Having described the subject matter of the present disclosure in detail and by reference to specific aspects, it is noted that the various details of such aspects should not be taken to imply that these details are essential components of the aspects. Rather, the claims appended hereto should be taken as the sole representation of the breadth of the present disclosure and the corresponding scope of the various aspects described in this disclosure. Further, it will be apparent that modifications and variations are possible without departing from the scope of the appended claims.

1. A process for producing low-carbon methanol, the process comprising:
upgrading a natural gas stream in a methanol production unit to produce a methanol reactor effluent, wherein the methanol production unit comprises a syngas synthesis section and a methanol synthesis section downstream of the syngas synthesis section;
introducing a captured CO₂ feed stream and a low-carbon hydrogen feed stream; of to the syngas synthesis section or to a syngas stream downstream of the syngas synthesis section and upstream of a methanol reactor of the methanol synthesis section, wherein:
at least a portion of the methanol reactor effluent comprises low-carbon methanol; and
the low-carbon methanol comprises a portion of total methanol in the methanol reactor effluent that is attributed to the introducing the captured CO₂ feed stream, the low-carbon hydrogen feed stream, or both to the methanol production unit based on a mass balance certification basis, an energy balance certification basis, or a trace-the-atom certification basis.

2. (canceled)

3. The process of claim 1, wherein the low-carbon hydrogen feed stream and the captured CO₂ feed stream are introduced to the methanol production unit at a molar ratio of low-carbon hydrogen to captured CO₂ of from 2 to 5.

4. The process of claim 1, wherein a weight ratio of the natural gas stream to the low-carbon hydrogen feed stream introduced to the methanol production unit is from 1 to 200.

5. The process of claim 1, wherein a weight ratio of the natural gas stream to the captured CO₂ feed stream introduced to the methanol production unit is from 0.1 to 40.

6. The process of claim 1, wherein the methanol production unit is an existing methanol production unit.

7. The process of claim 1, comprising introducing the captured CO₂ feed stream, the low-carbon hydrogen feed stream, or both to the syngas synthesis section of the methanol production unit.

8. The process of claim 1, wherein the syngas synthesis section comprises a primary reformer and a secondary reformer downstream of the primary reformer, and the process comprises introducing the captured CO₂ feed stream, the low-carbon hydrogen feed stream, or both to the syngas synthesis section downstream of the primary reformer and upstream of the secondary reformer.

9. The process of claim 1, wherein upgrading the natural gas stream in the methanol production unit to produce the methanol reactor effluent comprises:
hydrodesulfurizing the natural gas stream in a hydrodesulphurization unit to produce a desulphurized natural gas stream;
reforming the desulphurized natural gas stream in the presence of steam in a primary reformer downstream of the hydrodesulphurization unit to produce a primary reformer outlet stream;
reforming the primary reformer outlet stream in the presence of oxygen in a secondary reformer downstream of the primary reformer to produce a syngas stream comprising CO₂, CO, and H₂; and
converting the syngas stream in the methanol reactor disposed downstream of the secondary reformer to produce the methanol reactor effluent.

10. The process of claim 9, comprising introducing the captured CO₂ feed stream, the low-carbon hydrogen feed stream, or both to the secondary reformer or combining the captured CO₂ feed stream, the low-carbon hydrogen feed stream, or both with the primary reformer outlet stream upstream of the secondary reformer.

11. The process of claim 9, comprising introducing the captured CO₂ feed stream, the low-carbon hydrogen feed stream, or both to the methanol production unit downstream of the secondary reformer.

12. The process of claim 9, further comprising:
compressing the syngas in a syngas compression unit disposed downstream of the secondary reformer and upstream of the methanol reactor to produce a pressurize syngas stream;
passing the pressurized syngas stream to the methanol reactor; and
introducing the captured CO₂ feed stream, the low-carbon hydrogen feed stream, or both to the methanol production plant downstream of the secondary reformer and upstream of the syngas compression unit.

13. The process of claim 12, comprising:
combining the captured CO₂ feed stream, the low-carbon hydrogen feed stream, or both with the syngas downstream of the secondary reformer to produce a combined syngas stream;
compressing the combined syngas stream to produce a compressed combined syngas stream; and
passing the compressed combined syngas stream to the methanol reactor.

14. The process of claim 9, comprising passing only the low-carbon hydrogen feed stream to the methanol production unit downstream of the primary reformer and upstream of the secondary reformer, downstream of the secondary reformer and upstream of a syngas compression unit, downstream of the syngas compression unit and upstream of the methanol reactor, or combinations of these locations.

15. The process of claim 14, wherein a first portion of the low-carbon H₂ feed stream is combined with the primary reformer outlet stream upstream of the secondary reformer, and a second portion of the low-carbon H₂ feed stream is combined with the syngas stream downstream of the secondary reformer and upstream of the methanol reactor.

16. The process of claim 1, wherein a proportion of the low-carbon methanol in the methanol stream is from 0.01 wt. % to 40 wt. % based on total mass of methanol in the methanol stream.

17. The process of claim 1, wherein the low-carbon hydrogen feed stream comprises one or more of the following:
green hydrogen produced from water electrolysis using renewable energy sources;
blue hydrogen produced from fossil fuels with carbon capture and sequestration;
pink hydrogen produced from water electrolysis powered by nuclear energy;
turquoise hydrogen produced from methane pyrolysis;
hydrogen produced from a process of splitting of H₂S;
hydrogen produced from flue gas or waste gas with low-carbon emissions; or combinations thereof.

18. The process of claim 1, wherein the captured CO₂ feed stream comprise one or more of the following:
CO₂ directly captured from the atmosphere;
CO₂ produced from biogenic sources;
CO₂ captured from industrial point sources;
or combinations thereof.

19. A process of producing sustainable hydrocarbon fuels through fluidized catalytic cracking (FCC), the process comprising:
producing the low-carbon methanol according to the process of claim 1;
catalytically cracking a conventional FCC feed with an FCC catalyst in an existing FCC reactor to produce an FCC effluent comprising fuel and chemical components;
injecting the low-carbon methanol into the FCC reactor;
recovering the FCC effluent; and
certifying at least a portion of the FCC effluent as low-carbon fuel and chemical components, wherein the low-carbon fuel and chemical components are the fuel and chemical components attributed to injection of the low-carbon methanol to the FCC reactor based on a mass balance certification basis, an energy balance certification basis, or a trace-the-atom certification basis.

20. A process of producing sustainable hydrocarbon fuels in a dual FCC reactor system, the process comprising:
producing the low-carbon methanol according to the process of claim 1;
catalytically cracking a conventional FCC feed with a first FCC catalyst in a first FCC reactor to produce a first FCC effluent and used first FCC catalyst;
catalytically cracking the low-carbon methanol with a second FCC catalyst in a second FCC reactor to produce a second FCC effluent and a used second FCC catalyst, wherein the second FCC reactor is parallel to the first FCC reactor;
passing the first FCC effluent and the second FCC effluent to an FCC effluent separation system;
separating the first FCC effluent and the second FCC effluent in the FCC effluent separation system to produce at least on product stream comprising fuel and chemical components; and
certifying at least a portion of the fuel and chemical components in the at least one product stream as low-carbon fuel and chemical components, wherein the low-carbon fuel and chemical components are the fuel and chemical components attributed to fluidized catalytic cracking of the low-carbon methanol in the second FCC reactor based on a mass balance certification basis, an energy balance certification basis, or a trace-the-atom certification basis.

21. The process of claim 1, further comprising:
separating the methanol reactor effluent in a methanol purification unit disposed downstream of the methanol synthesis section to produce a methanol product stream and other constituents; and
recycling CO₂ from the other constituents back to the syngas synthesis section.
